(19) United States
(12) Patent Application Publication
(10) Pub. No.: US 2018/0256395 A1
(43) Pub. Date: Sep. 13, 2018

(54) SYSTEMS AND METHODS FOR THE INJECTION OF VISCOELASTIC FLUID

(71) Applicants: Luis Jose ESCAF, Baranquilla (CO); Luis Carlos ESCAF, Barranquilla (CO)

(72) Inventors: Luis Jose ESCAF, Baranquilla (CO); Luis Carlos ESCAF, Barranquilla (CO)

(21) Appl. No.: 15/980,144

(22) Filed: May 15, 2018

Related U.S. Application Data
(63) Continuation-in-part of application No. 15/429,863, filed on Feb. 10, 2017.

(51) Int. Cl. A61F 9/00; A61F 9/007; A61M 3/02

(52) U.S. Cl. CPC A61F 9/0017; A61M 31/00; A61M 3/0258; A61F 9/00745

(57) ABSTRACT

A system that provides for multiple viscoelastic container support and adjustment of a less full container with a fuller container to facilitate, for example, a continuous supply of a protective viscoelastic fluid into the anterior chamber of the eye with minimal operator disruption. The system provides for delivering viscoelastic material to an eye though use of a controlled driver driving a transmission that is in driving communication with an actuator for forcing viscoelastic fluid material from one of a plurality of containers. A viscoelastic material support assembly, which includes a multiple viscoelastic material container retention supporting structure and an adjustment mechanism for adjusting the retention supporting structure relative to the actuator into an injection capable setting, operates to supply a desired amount of viscoelastic material to the eye chamber. The system includes inputs, automated corrections, and visual interaction. Also provided are methods for using the system.

### BACKGROUND

The present invention generally relates to devices and procedures for the injection of viscoelastic fluid, with an example of such an injection being the supply of viscoelastic fluid to an eye such as during cataract surgery.

A cataract is a clouding of the lens inside the eye, causing vision degradation or loss that cannot be corrected with glasses, contact lenses or corneal refractive surgery like laser in-situ keratomileusis (LASIK). There are, however, surgical procedures for cataracts. In cataract surgery, the lens inside your eye that has become cloudy is removed and replaced with an artificial lens (called an intraocular lens, or IOL).

One procedure for removing the cloudy lens, called phacoemulsification or “phaco,” involves breaking up the cloudy lens into small pieces, which are then gently removed from the eye with suction. After all the remnants of the cloudy lens have been removed from the eye, the cataract surgeon inserts a clear intraocular lens, positioning it securely behind the iris and pupil, typically in the same location your natural lens occupied.

To perform the phaco procedure, the surgeon typically uses an ultrasonic surgical device consisting of an ultrasonically driven handpiece, an attached cutting tip (“phaco-tool”), an irrigating sleeve and an electronic control console. The handpiece assembly is connected to the control console through an electrical cable and a flexible tube. During the phaco procedure, the control console varies the power level transmitted by the handpiece to the cutting tip, and the flexible tubing is used to supply irrigation fluid to and draw aspiration from the eye through the handpiece. The operative part of the handpiece is a centrally located, hollow resonating bar or horn directly attached to piezoelectric crystals. The crystals supply the required ultrasonic vibration needed to drive both the horn and the attached cutting tip during phacoemulsification and are controlled by the console. The crystal/horn assembly is suspended with a hollow body or shell of the handpiece by flexible mountings. The handpiece body terminates in a reduced diameter portion or nosecone at the body's distal end. The nosecone is externally threaded to accept the irrigation sleeve. Similarly, the horn bore is internally threaded at its distal end to receive the external threads of the cutting tip. The irrigation sleeve also has an internally threaded bore that is screwed onto the external threads of the nosecone. The cutting tip is adjusted so that the tip extends only a predetermined amount past the open end of the irrigation sleeve.

In use, the ends of the cutting tip and irrigating sleeve are typically inserted into a small incision in the cornea. The cutting tip is ultrasonically vibrated by the crystal-driven ultrasonic horn, thereby emulsifying the selected lens tissue. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip and horn bores and the aspiration line and into a collection device. The aspiration of emulsified tissue is aided by a saline flushing solution that is injected into the surgical site through the small annular gap between the inside surface of the irrigating sleeve and the cutting tip.

It has been found that using such an ultrasonically driven handpiece in phaco procedures can cause damage to the eye. Particularly, it has been found that the ultrasonic energy used to emulsify the cloudy tissue of the lens can cause damage to the endothelial cells of the cornea.

Corneal endothelial help balance the flow of fluid into and out of the cornea, which helps the cornea remain transparent and therefore very important to clear vision. It is believed that the ultrasound energy used during phacoemulsification causes endothelial cell loss and/or damage during surgery and it also causes endothelial cell loss at a higher than normal rate for 10 years following the surgery. Since the endothelial cells of the cornea do not reproduce when damaged, endothelial cell loss can produce a range of different problems, from corneal edema to corneal descompensation or Bullous Keratopathy, in which the cornea loses its transparency resulting in the loss of visual acuity.

A solution to this problem has been to inject viscoelastic materials into the eye to maintain the stability of the anterior chamber of the eye, thus preventing its collapse during the procedure, and also to protect the corneal endothelium. The viscoelastic materials, sometimes referred to as ophthalmic viscoelastic devices (OVDs), have a viscous gel-like composition, which is used to coat the chambers of the eye in order to protect sensitive tissue in particular, endothelial cells, from trauma caused by the ultrasonic energy used in a phaco procedure. There are many such viscoelastic materials available today including, for example, Viscoat® and Healon®. A description of the types of viscoelastic materials utilized and methods utilized is given in U.S. Pat. No. 5,358,473, which is incorporated herein by reference in order to provide disclosure for the types of viscoelastic materials and the procedures commonly used to administer them. It has been found that the use of viscoelastic materials during a phaco procedure can reduce the incidence of endothelial cell damage both during and after the cataract surgery.

The common approach to using viscoelastic materials during a phaco procedure is to inject the fluids into the eye chambers by means of a hand held syringe or cannula. Because the flow characteristics and viscosity of the viscoelastic materials vary to some degree depending on factors such as the composition of the material, the temperature of the material, and the geometry of the injection apparatus, a manually-operated syringe is commonly used to enable direct physician control of the injection rate of the viscoelastic material into the eye.

The viscoelastic material is typically manually injected into the eye at the beginning of the surgical procedure to begin the phaco procedure. However, during surgery, the constant irrigation/aspiration and use of ultrasonic power to emulsify the target cloudy tissue tends to wash the viscoelastic material away from the sensitive tissue (e.g., endothelium) in the eye. This causes the endothelium to be more susceptible to short and long-term damage as described above.

In order to prevent such damage to the endothelium, the surgeon needs to add or direct an assistant to periodically add more viscoelastic material to the eye during the phaco procedure. This requires the surgeon to visually monitor the amount of viscoelastic material physically present in the eye during the surgery and actually stop the surgery so that a syringe or needle can be re-inserted into the eye whenever he/she decides more viscoelastic material needs to be added. The distraction from the surgical procedure to observe the viscoelastic material and the potentially repeated interruptions to re-insert a viscoelastic syringe or needle into the eye is highly undesirable, and can increase the risk of infection in the eye. In addition, since the decision as to whether more viscoelastic material should be added depends solely on the judgment and experience of the surgeon and/or the surgeon's assistant, such phaco procedures are subject to error and inconsistency from surgery to surgery and surgeon to surgeon.

One method for addressing the problems associated with repeated re-insertions of a syringe or needle into the eye each time additional viscoelastic materials need to be injected is disclosed in U.S. Pat. No. 6,254,587 (the '587 patent), which is incorporated herein by reference. The '587 patent teaches a method for dispensing viscous fluid into the eye during surgery upon demand, without the need to re-insert a viscoelastic syringe or needle each time the viscoelastic materials are added. In particular, the '587 patent teaches using a dispensing means which includes, in part, a flexible diaphragm defining a first chamber filled with viscoelastic materials and a second chamber filled with pressurized air. The second chamber is connected to a phacoemulsification machine adapted for providing a constant controlled source of air pressure. The first chamber is connected to a conduit in the phacoemulsification handpiece which includes a means for dispensing the viscoelastic material to the eye and proximate the needle tip of the handpiece. The means for dispensing the viscoelastic material includes a normally closed valve disposed on the housing of the handpiece such that when the surgeon wants to inject additional viscoelastic material into the eye during surgery, he/she needs to open the valve. Upon opening the valve, the constant source of air pressure will push the viscoelastic material through the conduit into the handpiece whereby the material is injected into the eye proximate the needle tip.

Although the method disclosed in the '587 patent provides a method for dispensing viscoelastic materials without having to sporadically and repeatedly re-insert a needle into the eye during surgery, it still requires and depends solely on the surgeon's ability to visually observe the amount of viscoelastic materials in the eye and the surgeons judgment on whether additional viscoelastic materials need to be injected, thereby being susceptible inaccuracy due to differences in visual capabilities and experiences from surgeon to surgeon. Furthermore, the reliance on the surgeon to gauge, in a dynamic process where focused visualization can be difficult, the viscoelastic fluid status, both from the standpoint of how much is currently in the eye and how much is being fed to the eye upon trigger initiation (and the period following triggering), can lead to inadequate supply or even an oversupply situation, with the resultant problems associated with each. Viscoelastic material is injected into the eye at the beginning of a surgical procedure and often times during the procedure to make up for lost viscoelastic material as due to saline irrigation of the phaco broken up cataract pieces. Thus, there is a need for a ready supply during the procedure, and any disruption or delay in maintaining a supply on-demand can be problematic.

Thus, there is a need for an improved system and method for administering viscoelastic materials into the eye, wherein the decision as to the amount and timing of viscoelastic materials injected into the eye does not depend solely on the surgeon's visual capabilities and judgment. There is also a need for an improved system that can remove the potential for a delay or disruption in an on-demand supply of the viscoelastic material.

### SUMMARY

It is to be understood that both the following summary and the following detailed description are exemplary and explanatory only and are not restrictive, as claimed. In one aspect, provided are methods and systems for dispensing viscoelastic materials into an eye during surgery without relying solely on the visual acuity and experience of the surgeon to determine if and when viscoelastic materials should be added. Instead, provided are systems and methods that automatically dispense viscoelastic materials into the eye based on a measurement of one or more operating conditions or parameters such as intraocular pressure of the eye, feed line pressure, injection speed, and injection volume.

In an aspect, the system comprises a controller, an intraocular pressure sensor, and a phacoemulsification handpiece, wherein the controller comprises a viscoelastic materials pump and a processor for activating and deactivating the viscoelastic materials pump based on a measurement of the intraocular pressure of the eye by the intraocular pressure sensor wherein, upon activation, the pump dispenses viscoelastic materials into the eye through the handpiece.

In an aspect, the system comprises a controller, an intraocular pressure sensor, a phacoemulsification handpiece, and a maintainer, wherein the controller comprises a viscoelastic materials pump and a processor for activating and deactivating the pump based on a measurement of the intraocular pressure of the eye by the intraocular pressure sensor wherein, upon activation, the pump dispenses viscoelastic materials into the eye through the maintainer.

Accordingly, based on intraocular pressure measurement, a determinant can be made as to whether or not more viscoelastic material should be injected into the eye. In other words, when viscoelastic material is needed. Under an aspect of the invention there is also monitored/determined (either in conjunction with an intraocular pressure measurement or independently and without reliance on an intraocular pressure measurement) one or more (preferably all) of the following: how much (volume) of viscoelastic material should be introduced, and has been introduced, the velocity of the viscoelastic material being introduced, and the conduit (non-intraocular) pressure of the viscoelastic material being introduced (which is related to the eventual intraocular pressure in the eye chamber).

In another aspect, the system further comprises a means for manually activating and deactivating the pump which is inclusive of one or both of a handpiece trigger and a hands-free trigger as in a foot pedal flow trigger switch.

In another aspect, the intraocular pressure sensor comprises an inductance-capacitance (LC) resonant circuit implanted in the anterior chamber of the eye, and wherein the controller comprises an intraocular pressure detector for calculating a measurement of the intraocular pressure based on the resonant frequency of the LC resonant circuit.

In another aspect, the intraocular pressure sensor comprises a pressure-sensitive nanophotonic structure implanted into the anterior chamber of the eye, the pressure-sensitive nanophotonic structure having an optical signature that changes as a function of the intraocular pressure of the eye.

In another aspect, the intraocular pressure sensor comprises a programmable intraocular pressure sensor system implant integrated on a single CMOS chip, wherein the CMOS chip comprises a micromechanical pressure sensor (MEMS) array, a temperature sensor, an antenna, a capacitive powering array, readout and calibration electronics, a microchip-based digital control unit, and an RF-transponder.

In another aspect, a system comprises a viscoelastic material dispensing system having a mechanical device for injecting viscoelastic material through a cannula into the eye, and an electronic system for measuring the intraocular pressure of the eye, and one or more (e.g., each) of the speed, line pressure and injected volume (e.g., a predetermined “plug” of viscoelastic material to be supplied) of the viscoelastic material being injected by the mechanical device into the eye. In the context of the present invention the term “one or more” is inclusive of all options under the “or more” as well as any of the potential sub-groups under the “or more” phrase.

An additional aspect features a viscoelastic fluid injection system having an ability to control the speed of injection of the viscoelastic fluid into the eye, the volume or amount of viscoelastic material to be fed (and being fed) into the eye, and the conduit pressure of the fluid that is being introduced in the anterior chamber of the eye (or other compartment where the viscoelastic material in injected). As an example, upon triggering activation of the injection system, as by a foot pedal accessible to a surgeon or an assistant of the surgeon, there is initiated an injection protocol that has been previously set up as to the velocity, pressure, and amount of injected viscoelastic material being fed to the eye (either with or without real time feedback from the aforementioned controller, inclusive of controller coordination with the intraocular pressure monitoring system).

In other words, an aspect of the invention features an injection system that is versatile during an operation in making suitable adjustments as to viscoelastic material feed during an operation, but also provides for a physician preset of a desired velocity, pressure and flow volume to be fed to the chamber of the eye both at initiation of a procedure as well as during the procedure if deemed helpful. In this regard, a suitable surgeon, or surgery assistant, interface, as in a touch screen, is provided for easy setting and monitoring of the viscoelastic injection settings and status (e.g., status as to flow injection presently on going, adjustment capability as to flow parameters based on one or more measurements of intraocular pressure, injection speed, and/or injection flow volume, viscoelastic material current supply monitoring status, status as to the availability of alternate viscoelastic supply sources). In addition to maintaining one or more of a preset desired volume, pressure, flow speed of the injected viscoelastic material, there is provided, under an aspect of the present invention, a shutdown component as in a maximum pressure sensor. An example being an in line pressure sensor that can rapidly pick up a line blockage condition and trigger an immediate shut down in the system (alternatively the shutdown system can be based on alternate sensor types as in one, or more, or all of sensors that detect the pump motor torque levels, viscoelastic fluid line speed and/or volume flow rate when in active mode, or such other sensing can be used as a supplement to the pressure sensing for failsafe purposes). Supplementation in this regard can be an added sensing requirement for shut down (two sensed conditions met for shutdown, or an alternative supplementation wherein either type of sensed condition can trigger shutdown).

A further aspect of the present invention's scope includes a system for delivering viscoelastic material to an eye, that includes a driver, a transmission in driving communication with the driver, an actuator in driving communication with the transmission, and a viscoelastic material support assembly which includes a multiple viscoelastic material container retention supporting structure and an adjustment mechanism for adjusting the retention supporting structure relative to a transmission driven actuator into an injection capable setting.

An additional aspect of the present invention's scope includes a controller with a processor that activates the driver following the adjustment mechanism's alignment of one of a plurality of container supports or cradles supported by the retention supporting structure into the injection capable setting.

An additional aspect of the present invention's scope includes an arrangement in the adjustment mechanism that includes a manual push-pull member connected with the container retention supporting structure.

An additional aspect of the present invention's scope includes an arrangement wherein the container retention supporting structure includes a plurality of cradles configuring to receive a respective one of a plurality of viscoelastic material containers.

An additional aspect of the present invention's scope includes an arrangement wherein the cradles are dimensioned to receive syringe shaped viscoelastic material containers having plungers extending out from fluid containing cylinders.

An additional aspect of the present invention's scope includes an arrangement wherein the retention supporting structure includes a plurality of support blocks that include slide tracking for side-to-side adjustment based on positioning of the push-pull member.

An additional aspect of the present invention's scope includes an arrangement wherein each support block has a universal configuration relative to another support block of the support block set.

An additional aspect of the present invention's scope includes an arrangement that includes a housing wherein the housing includes a slide track on which the container supports structure can slide.

An additional aspect of the present invention's scope includes an arrangement wherein the actuator includes a base part and a projection part wherein the projection part is configured for abutment with a plunger portion of a viscoelastic material container aligned by the adjustment mechanism for emptying.

An additional aspect of the present invention's scope includes an arrangement wherein the transmission includes a linear screw assembly to which the actuator is movably connected.

An additional aspect of the present invention's scope includes an arrangement wherein the actuator is adjustable in a longitudinal direction transverse to the side-to-side slide direction of a container retention supporting structure.

An additional aspect of the present invention's scope includes an arrangement that features an encoder, with the controller having an actuator position monitoring unit that is configured to monitor actuator position and conveys that information as to the fill level of one or each of the containers to the controller.

An additional aspect of the present invention's scope includes an arrangement that features a visualization system in communication with the controller and which visualization system has a fill level designation section that conveys a virtual fill level depiction of one or more of the containers (as determined, for example, by the above referenced encoder monitoring in conjunction with monitored knowledge as to slide positioning of the containers (e.g., slide contact signals to the controller), following controller initiation with a preset number of full containers). In this way, there can be provided for a system wherein the fill level designation section conveys the fill level of each of multiple containers supported by the container retention supporting structure.

An additional aspect of the present invention's scope includes an arrangement that features a controller in communication with the driver, as in an electric motor driver, with the interfacing between the controller and motor being inclusive of torque level data of the motor and conveys that torque information as to indicate, for example, pressure levels of viscoelastic fluid being fed by actuator movement by the actuator.

An additional aspect of the present invention's scope includes an arrangement wherein the retention supporting structure includes multiple cradles for supporting viscoelastic containers which are slideable in a longitudinal direction, and wherein the retention supporting structure includes base blocks which supports said cradles and include a slide configuration for sliding in a side-to-side direction which is perpendicular to the longitudinal direction.

An additional aspect of the present invention's scope includes a system for delivering viscoelastic material that features driving means for providing a driving force, an actuator which receives the driving force, supporting means for supporting a plurality of containers, as well as an adjustment means for adjusting the supporting means to multiple positions to provide for the positioning of a desired container for actuator adjustment and viscoelastic material injection.

An additional aspect of the present invention's scope includes an arrangement that features a visualization system showing which container is adjusted for immediate dispensing and the relative fill level for each of the multiple containers.

An additional aspect of the present invention's scope includes an arrangement that features a visualization system configured to show the relative fill level of the multiple containers and the adjustment means being adjustable to place a fuller container in place of a less full container in alignment with an actuator of the actuation means.

An additional aspect of the present invention's scope includes a method of delivering viscoelastic material of a system for delivering viscoelastic material to an eye (which system includes a driver, a transmission in driving communication with the driver, an actuator in driving communication with the transmission, and a viscoelastic material support assembly which includes a multiple viscoelastic material container retention supporting structure and an adjustment mechanism for adjusting the retention supporting structure relative to a transmission driven actuator into an injection capable setting). An aspect falling under the scope of the present invention includes the method of delivering that includes a step of adjusting the retention support structure as to move a less full viscoelastic material container from an actuator alignment position to another location, while bringing a fuller viscoelastic material container into the actuator alignment position.

Additional embodiments and advantages will be set forth in part in the description which follows. The advantages will be realized and attained by means of the elements and combinations particularly pointed out in the appended claims. It is to be understood that both the foregoing general description and the following detailed description are exemplary and explanatory only and are not restrictive, as claimed. Moreover, references to “embodiments” or “aspects” (or like terminology) of the present invention are not intended in the present application to be limiting to the same embodiment being then discussed. For example, the particular features, structures, materials, methods, or characteristics may be combined in any suitable manner in one or more embodiments.

### BRIEF DESCRIPTION OF THE FIGURES

The invention and the following detailed description of certain embodiments thereof may be understood with reference to the following figures:

FIG. 1 is a schematic diagram of an exemplary system in accordance with the invention showing a means for injecting viscoelastic materials into an eye through a phacoemulsification handpiece.

FIG. 1A is a schematic diagram similar to that of FIG. 1, but of an additional exemplary system in accordance with the invention featuring a hand-less mechanical injection system trigger and/or hand-less injection start triggering means.

FIG. 2 is a schematic diagram of an exemplary system in accordance with the present invention showing a means for injecting viscoelastic materials into an eye through a maintainer.

FIG. 3 shows an exemplary embodiment of a stand-alone, single container viscoelastic material injection system in accordance with the invention.

FIG. 4 shows a functional block schematic illustration of a viscoelastic system aspect under the present invention.

FIG. 5 shows an exemplary embodiment of a stand-alone, multi-container viscoelastic material(s) injection system in accordance with the present invention with the activator in a retracted position and the loader in a centralized position relative to a container set with each container in a full state.

FIG. 6 shows the stand-alone viscoelastic materials injection system of FIG. 5 from an alternate perspective.

FIG. 7 shows the stand-alone viscoelastic materials injection system of FIG. 6 with the cover removed and each container in a fully dispensed or emptied state with the activator retracted for full container replenishment.

FIG. 8 shows a top plan view of the injection system of FIG. 7 (containers in an emptied state).

FIG. 9 shows a side elevational view of the injection system of FIG. 8.

FIG. 10 shows a front view of the injection system of FIG. 8.

FIG. 11A shows an enlarged view of the front region of the injection system of FIG. 5 showing the adjustable multi-container support assembly as well as a plurality of flexible injection conduits that are shown in cut-away at a location downstream from their screw cap threaded container connection ends.

FIG. 11B shows, in cut away fashion, a downstream portion of the conduits shown in FIG. 11A including a multi-conduit-to-single conduit outlet manifold adapter that can be, for example, a component retained within the handpiece of FIG. 1, or as a maintainer cannula component of FIG. 2, such that the one output viscoelastic material conduit can be readily positioned for, for example, eye anterior chamber injection or can be a component outside of the handpiece phaco-tool injection site as in an injection needle provided in its own independent eye incision location.

FIG. 12 shows one of the plurality of viscoelastic material container supports or container cradles shown in FIG. 11A which is sized for releasable securement of a predetermined diameter(s) container.

FIG. 13 shows one of the plurality of sliding support blocks or receptors with each slidingly receiving, in a top region, a respective cradle, and with each featuring an adjustment rod aperture that receives an adjustment rod as to enable transverse (e.g., hand) adjustment to different container actuator alignment locations.

FIG. 14A shows a view similar to FIG. 5, but with the actuator having been moved longitudinally to achieve an emptying of the central positioned container amongst the three viscoelastic material containers shown, with the other two still in a full state.

FIG. 14B shows a view similar to FIG. 14A, but with the actuator having been retracted and the adjustment mechanism for adjusting the retention supporting structure having been adjusted to move the empty container out of alignment with the actuator and a still full container (leftmost one featured in this sequence) adjusted into alignment with the non-adjusted actuator.

FIG. 14C shows a view similar to FIG. 14B, but with two of the containers that have been emptied by the actuator shifted by the adjustment mechanism following retraction of the actuator as to position the last full container (the rightmost one) in alignment with the retracted actuator.

FIG. 15 shows a schematic display of a visual display and input interface wherein a user can input and monitor various aspects of viscoelastic fluid material injection and which further features a maximum level shut down display.

FIG. 16A shows a flow logic diagram for illustrating controller configuration for providing for operator desired presetting and monitoring of the same.

FIG. 16B shows a flow logic diagram for illustrating the controller configuration for providing for maximum setting determination and system shutdown.

FIG. 16C shows a flow logic diagram for illustrating controller configuration for achieving multi-container monitoring and position adjustment control.

FIG. 17 illustrates a phaco-tool with associated saline irrigation and suction conduits in a first eye incision and a viscoelastic material supply line inserted into a second (independent to first) incision in the eye for controlled supply of viscoelastic material in accordance with aspects of the invention.

### DETAILED DESCRIPTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting, but to provide an understandable description of the invention.

The terms “a” or “an,” as used herein, are defined as one or more than one. The term “another,” as used herein, is defined as at least a second or more. Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes. In addition, this disclosure uses certain terms relating to exemplary phacoemulsification surgery devices and systems. For example, the terms “handpiece”, “controller”, “pump”, “probe”, “needle”, and “cannula” are used herein for convenience, and are not intended to limit the scope of the disclosure to a particular phacoemulsification device or system.

Disclosed are components that can be used to perform the described methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. The present methods and systems may also take the form of web-implemented computer software. Any suitable computer-readable storage medium may be utilized including hard disks, optical storage devices, or magnetic storage devices.

Embodiments of the methods and systems are described below with reference to block diagrams and flowchart illustrations of methods, systems, and apparatuses. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

As described in the background, a cataract is a clouding of the lens inside the eye. One procedure for cataract surgery is phacoemulsification. Phacoemulsification involves the breaking up of the cloudy lens into small pieces, which are then gently removed from the eye with suction. In phacoemulsification surgery, the surgeon uses a handpiece having an ultrasonically driven horn and cutting tip. The handpiece is connected to a controller through an electrical cable. During the phacoemulsification procedure, the controller varies the ultrasonic power level transmitted by the handpiece to the horn and cutting tip for emulsifying the cloudy lens tissue. The handpiece is also connected to a flexible tube used to provide irrigation fluid to the eye. The handpiece is typically configured to dispense the irrigation fluid proximate the cutting tip. The handpiece is also connected to a flexible tube for aspirating the irrigation fluid and any emulsified eye tissue. The handpiece is typically configured to aspirate through a separate sleeve inserted into the eye. Further, the handpiece is connected to a flexible tube for dispensing viscoelastic materials into the eye. The viscoelastic materials are used to protect sensitive eye tissue during the surgery. The handpiece is configured to inject the viscoelastic materials into the eye proximate the needle tip.

As described in the background, the viscoelastic material is typically inserted into the eye at the beginning of the surgery. However, during the surgery, the ultrasonic vibrations, irrigation and aspiration activities tend to wash away the viscoelastic materials, thereby leaving the sensitive eye tissue, e.g., endothelial cells less protected. As a result there typically needs to be periodically added viscoelastic materials to the eye during the surgery.

As described in the background, the known methods for dispensing or injecting the viscoelastic materials into the eye require the surgeon to visually inspect the eye during surgery, observe the amount of viscoelastic material present in the eye, and make a judgment as to whether more viscoelastic material needs to be added. The additional viscoelastic material is often added by way of a syringe having a needle manually inserted into the eye. In such a case, the addition of viscoelastic material to the eye depends solely on the visual inspection (observations) and judgment of the surgeon. This not only distracts the surgeon from other aspects of the surgery including, for example, cutting and aspirating the eye tissue, it also tends to provide different levels of accuracy from surgeon to surgeon. Thus, there is a need for an improved system and method for administering viscoelastic materials into the eye that enables the surgeon to pay more attention to the cutting and emulsifying aspects of the surgery and does not rely solely on the surgeon's visual acuity and judgment.

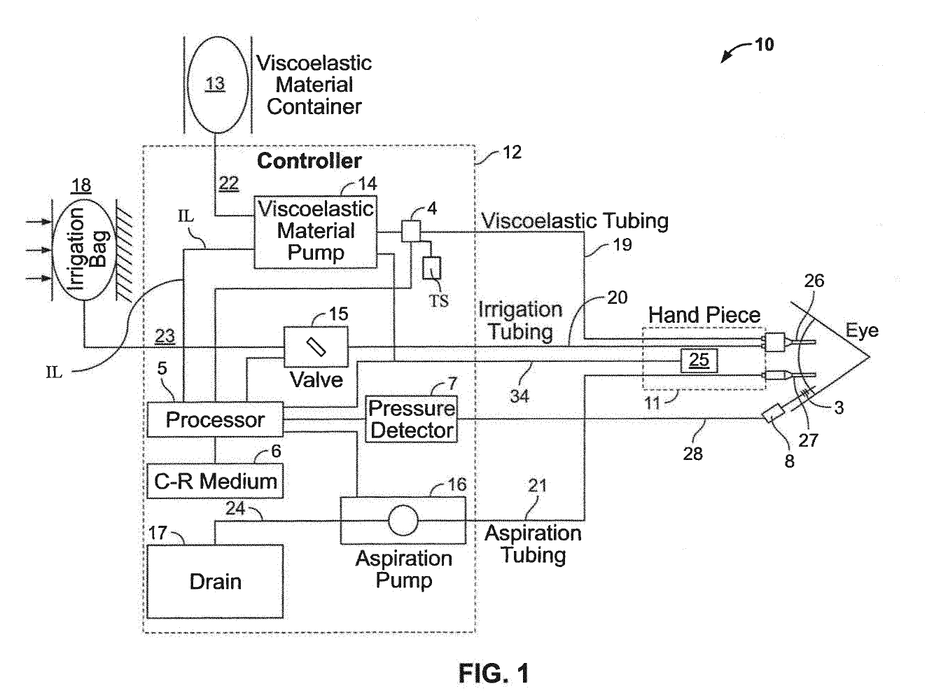

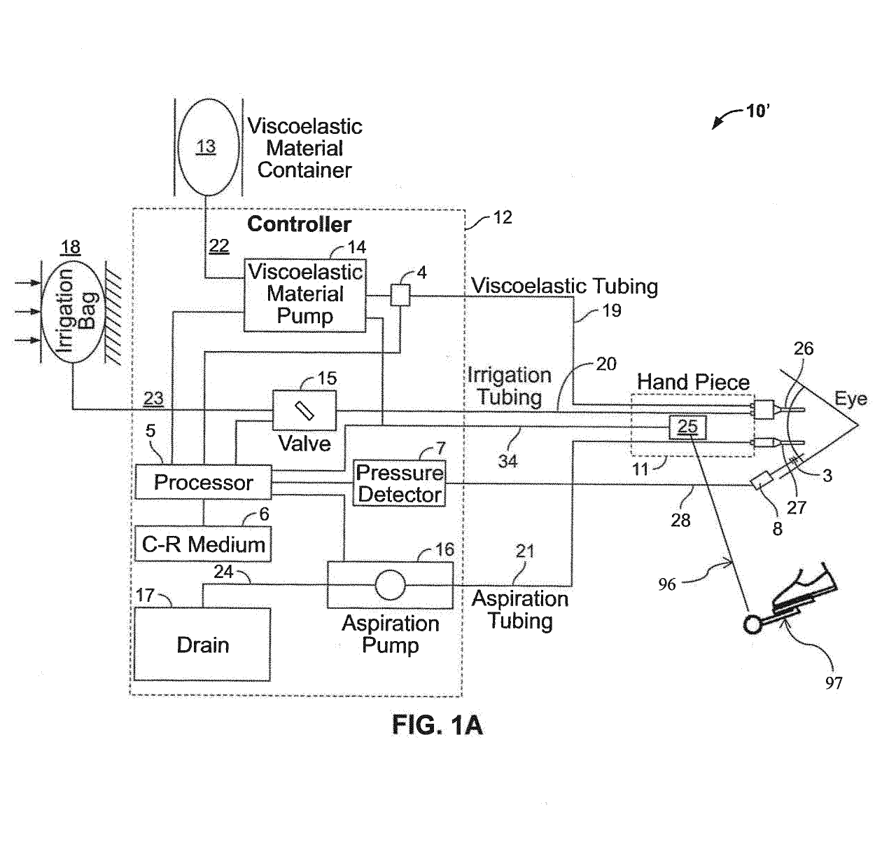

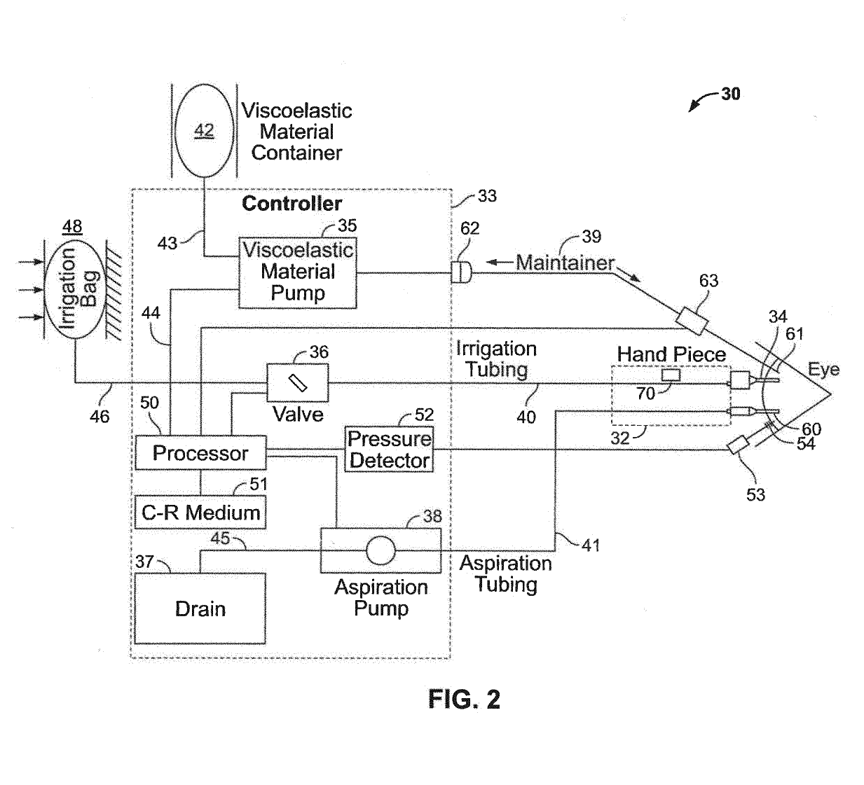

Referring now to FIGS. 1 and 2, embodiments of systems in accordance with the present invention are shown. For simplicity, the exemplary systems shown in FIGS. 1 and 2 are directed to systems and methods useful in ophthalmic surgical procedures, and are similar in structure and operation except that they comprise different means for dispensing or injecting viscoelastic materials into the eye. FIG. 1 shows an exemplary means for dispensing and injecting viscoelastic materials into an eye through a phacoemulsification handpiece, whereas FIG. 2 shows an exemplary means for dispensing and injecting viscoelastic materials into an eye through a maintainer. As will be explained in more detail below, various other structures, systems and methods are contemplated and thus it should be understood that the systems of FIGS. 1 and 2 are only exemplary embodiments of systems and methods in accordance with the present invention.

In FIG. 1, there is shown a system 10 comprising a controller 12 and a handpiece 11, wherein handpiece or handset 11 is shown having a needle (or cataract break-up means or “phaco-tool”) 26 and an aspiration tube 27, each inserted into an eye (not part of system 10). Needle 26 includes an ultrasonically-driven horn and a needle tip configured to cut eye tissue, and may be of any conventional suitable design heretofore used in phacoemulsification handpieces. Similarly, as in conventional phacoemulsification handpieces, handpiece 11 is electrically connected (not shown) to controller 12 such that controller 12 can vary the ultrasonic power delivered by handset 11 through needle 26 for the emulsification of tissue in the eye. Thus, similar to conventional phacoemulsification handpieces, the tip of needle 26 upon vibrating at ultrasonic frequencies is capable of cutting or fragmenting eye tissue. Thus, needle 26 provides means for radiating ultrasonic energy into an eye in order to cut, fragment or emulsify tissue, depending on the particular surgical procedure being conducted.

Controller 12 comprises a processor 5 electrically connected to a computer-readable medium 6, an aspiration pump 16, an intraocular pressure detector 7, a valve 15, a viscoelastic material pump 14, and an electrical switch 25 on handpiece 11. Electrical switch 25 can be manually operated by a surgeon to switch between an on and an off state. When switch 25 is switched to the on state, a signal is transmitted to processor 5 to direct processor 5 to activate viscoelastic pump 14. When switch 25 is switched to the off state, a signal is transmitted to processor 5 to deactivate the viscoelastic pump 14.

Computer readable medium 6 stores programs for operating controller 12 including, but not limited to, a program for controlling the operation of viscoelastic material pump 14, a program for controlling the operation of aspiration pump 16, a program for controlling the operation of pressure detector 7, a program for controlling the operation of valve 15, a program for controlling the ultrasonic power delivered by handpiece 11 to needle 26, and a program for processing an electrical signal from electrical switch 25 on handpiece 11. Processor 5 can call and execute these programs from computer-readable medium 6, as needed.

Viscoelastic material pump 14 is connected to a viscoelastic material container 13 through a tube 22 and to handpiece 11 through viscoelastic tube 19. Viscoelastic material container 13 can be any type of container suitable for holding and dispensing viscoelastic materials. Viscoelastic container 13 can be refillable or removable such that replacement viscoelastic materials can be added to container 13 or a replacement container can be attached to tube 22, as needed. Further, viscoelastic container 13 is configured such that when attached to tube 22 and upon activation of viscoelastic pump 14, viscoelastic material in container 13 can be drawn through tube 22 by the suction draw of the viscoelastic pump 14, and pumped to handpiece 11 through viscoelastic tube 19. Handpiece 11 can thereby inject the viscoelastic materials into the eye through needle 26. The viscoelastic material container 13 can contain any type of viscoelastic materials including, for example, Viscoat® and Healon®.

Valve 15 is connected to an irrigation bag 18 through a tube 23 and to handpiece 11 through an irrigation tube 20. Irrigation bag 18 can contain any type of irrigation fluid including, for example, a saline solution. Irrigation bag 18 is removable such that a replacement bag can be attached to tube 23, as needed. Irrigation bag 18 is configured such that when attached to tube 23 and upon the opening of valve 15, the irrigation fluid in irrigation bag 18 can be fed to through tube 23 to handpiece 11 and injected into the eye through needle 26.

Aspiration pump 16 is connected to handpiece 11 through aspiration tube 21, and to drain 17 through tube 24. Drain 17 can be any type of container for collecting aspirated fluids, materials and emulsified tissue from the eye. Upon activation of aspiration pump 16, fluids, materials and emulsified tissue in the eye can be drawn or vacuumed from the eye through sleeve 27 to aspiration tube 21 and fed to drain 17 through tube 24.

Pressure detector 7 is electrically connected through electrical cable 28 to an intraocular pressure-monitoring system 8 coupled to an intraocular pressure sensor 3 (shown inserted into the eye). The intraocular pressure sensor 3 is operable to generate pressure measurement data related to the intraocular pressure of the eye. The pressure measurement data is captured and transmitted by intraocular pressure-monitoring system 8 to intraocular pressure detector 7 over communication cable 28. Intraocular pressure detector 7, along with processor 5, use the data to compute a measured intraocular pressure of the eye.

It should be appreciated therefore that, in system 10, the means for measuring the intraocular pressure of the eye comprises an intraocular pressure sensor 3, an intraocular pressure monitoring system 8, a pressure detector 7 and a processor 5 communicating with a computer readable medium 6. It should be understood, however, that the present invention is not limited to the means for measuring the intraocular pressure of system 10. A system and method in accordance with the invention can comprise any means for measuring the intraocular pressure comprising any type of intraocular pressure sensor and any type of pressure monitoring system known in the art.

For example, a means for measuring the intraocular pressure in a system in accordance with the present invention could comprise a processor, a pressure monitoring system and an intraocular pressure sensor comprising an inductance-capacitance (LC) resonant circuit, wherein the LC resonance circuit has a resonance frequency that changes as a function of changes in the intraocular pressure, wherein the intraocular pressure monitoring system comprises a coil for sensing the changes in the resonance frequency and a means for transmitting data, based on the measure changes in resonance frequency, to the processor, which calculates the measured intraocular pressure based on the data.

As another example, a means for measuring the intraocular pressure in a system in accordance with the present invention could comprise an intraocular pressure sensor comprising a pressure-sensitive nanophotonic structure, a pressure monitoring system comprising an optical reader, wherein the optical reader optically excites the nanophotonic structure and detects the reflected light, whose optical signature changes as a function of the intraocular pressure. The optical signature data can then be processed to determine the measured intraocular pressure.

In yet another example, a means for measuring the intraocular pressure in a system in accordance with the present invention could comprise a programmable intraocular pressure sensor system implant integrated on a single CMOS chip, wherein the CMOS chip comprises a micromechanical pressure sensor (MEMS) array (i.e., an intraocular pressure sensor), and a pressure monitoring system comprising an antenna, a capacitive powering array, readout and calibration electronics, a microchip-based digital control unit, and an RF-transponder, wherein the pressure monitoring system can wirelessly communicate the intraocular pressure reading (or data related thereto) to a remote processor including, for example, a phacoemulsification controller for receiving the intraocular pressure measurement or for determining the intraocular pressure measurement based on the data, as the case may be.

In still yet another embodiment, the internal eye pressure could comprise means for extrapolating by way of an external change in eye surface topography, using, for example, exterior eye topography changes due to higher/lower interior pressure levels leading to an exterior surface curvature difference in the eye topography. In such a technique, standard eye topography measuring equipment as in that utilized in the aforementioned LASIK surgery, can be utilized and linked to, for example, the present inventions controller 12 for pressure monitoring. In such a case the intraocular pressure monitoring system 8 and pressure detector 7 would be replaced by a topography sensor as in one with a laser centering device that feeds information to processor 5 for internal eye pressure extrapolation from exterior eye contouring.

In all such embodiments, in accordance with the invention, the processing of the pressure sensor data captured by the pressure-monitoring system can be performed by a processor located in the intraocular pressure sensor, the pressure-monitoring system, or by a remote pressure detector and/or processor including, for example, a processor located in a controller for a phacoemulsification system.

System 10 can utilize the means for measuring the intraocular pressure to automatically dispense and/or inject viscoelastic materials into the eye during surgery. That is, in accordance with the invention, viscoelastic materials can be injected into the eye as a function of intraocular pressure. In operation, processor 5 calls the program for monitoring the intraocular pressure form computer-readable medium 6. During execution of the program, processor 5 directs pressure detector 7 to utilize intraocular pressure sensor 3 and pressure monitoring system 8 to obtain data related to the intraocular pressure of the eye. As described above, different methods for obtaining the data can be employed depending on the type and structure of intraocular pressure sensor 3 and pressure monitoring system 8. Once the data is returned to intraocular pressure detector 7, processor 5 can determine a measured intraocular pressure based on the data. The measured intraocular pressure level depends, in part, on the amount of viscoelastic material in the eye. The measured intraocular pressure is compared to a target pressure level, wherein the target pressure level is selected to be a level that provides for a desired amount of viscoelastic material to be present in the eye. Thus, if the measured intraocular pressure is below the target level, processor 5 activates viscoelastic materials pump 14, unless viscoelastic material pump 14 is already activated. Upon and during activation, viscoelastic materials pump 14 draws viscoelastic materials from container 13 and pumps the materials to handpiece 11 through viscoelastic tube 19. Handpiece 11 thereby injects the viscoelastic materials into the eye through needle 26, to thereby increase the measured intraocular pressure of the eye to a level indicative an appropriate amount of viscoelastic material in the eye to protect eye tissue during surgery. If the measured intraocular pressure level is at or above the target intraocular pressure level, the processor 5 deactivates the viscoelastic materials pump 14, unless the pump is already deactivated. As will be explained in greater detail below, the target intraocular pressure is at a level that is below a maximum pressure level that is set at a value where; if exceeded, (e.g., by a factor of safety) could result in potential eye damage (despite some degree of potential leakage of viscoelastic material through one or more eye incisions formed in the eye).

As a safeguard against the target pressure being set too low, or the means for measuring the intraocular pressure being inaccurate, system 10 provides for means to inject viscoelastic material on demand. This can be accomplished by manually operating switch 25 as on handpiece 11. Upon detecting signal switch 25 has been manually switched to the on state, processor 5 will activate viscoelastic pump 14, thereby providing viscoelastic materials to handpiece 11 in the same manner as described above. Upon detecting that switch 25 has been manually switched to the off state, processor 5 will deactivate viscoelastic pump 14, unless it is determined that that the intraocular pressure is below the target intraocular pressure level which, in that case, the processor 5 can be configured as not to deactivate viscoelastic pump 14 or can be configured to send out an auditory signal or the like to signal that the pressure is still deemed too low and seek confirmation of pump shut down desirability (as in another trigger implementation).

It should be appreciated that the target intraocular pressure level can be determined and set in a number of different ways and based on any number of factors. For example, the surgeon can determine the target pressure level based on his/her experience, the conditions of the surgery, the characteristics of the eye under surgery, the age of the patient, the gender of the patient, the health of the patient, and the type of viscoelastic materials being used in the surgery.

In an embodiment, the surgeon could enter the target pressure level through a keyboard interfacing with controller 12. In another embodiment, the surgeon could enter the target pressure level through an interface located on handset 11. In yet another embodiment, the surgeon could enter the target pressure level from a terminal, a handheld device that communicates with the controller 12 through a wireless interface or voice communicator. In yet another embodiment, the surgeon could enter factors (such as the above mentioned age, health, gender, viscoelastic material, etc.) into controller 12, wherein controller 12 will calculate the target pressure level.

It should also be appreciated that the target pressure level can be adjusted or changed during surgery. Therefore, it is contemplated that, if the surgeon determines that the target pressure level is not causing the controller 12 to activate the viscoelastic pump 14 to provide enough viscoelastic material during surgery; the system 10, in an exemplary embodiment of the invention, will have means for enabling the surgeon to adjust or change the target level on demand. For example, in an embodiment, the handpiece 11 can have a set of buttons, one for increasing the target pressure value and the other for decreasing the target pressure value. The buttons can be electrically connected to controller 12 or alternatively, communicate with controller 12 through other means including, for example, a wireless interface. Controller 12 can then use the new or changed target pressure level when it runs the programs for comparing the measured intraocular pressure to the target pressure for controlling the operation of the viscoelastic material pump 14.

In another embodiment, the handpiece 11 can have an interface including, for example, a keyboard or touch screen for inputting changes (in addition to initial settings) to the value of the target pressure level. The changes can then be communicated to the controller 12 through an electrical connection (not shown) between the handpiece 11 and the controller 12 or by any other communication means including, for example, a wireless connection or voice command.

In another embodiment, the controller 12 can have an interface for inputting changes to the target pressure level. The interface could be, for example, a touch screen or a set of buttons, similar to that described above for embodiments of the handpiece 11.

By providing the means for automatically adding viscoelastic materials into the eye based on intraocular pressure, system 10 reduces the surgeon's distraction from other aspects of the surgery and provides for a more consistent and accurate handling of viscoelastic materials in the eye from surgeon to surgeon. In addition, by enabling the surgeon to request through switch 25 on handpiece 11, the injection of additional viscoelastic material into the eye or a reduction of viscoelastic material flow into the eye, system 10 provides for addition protection and flexibility for making sure the sensitive tissue in the eye is well-protected during surgery.

In another embodiment, system 10 may also control the injection viscoelastic materials into the eye based on a presetting of the injection speed and/or volume. That is, viscoelastic materials can be injected into the eye as a function of intraocular pressure, injection speed, injection volume, or some combination thereof. For example, in an aspect of the invention rather than reliance on an intraocular pressure-monitoring system to provide guidance as to the flow of viscoelastic material to the eye, the surgeon's preset values can be utilized in conjunction with system controlled and monitored input relative to those preset values as the sole guidance, although preferably with the aforementioned flexibility of adjusting preset values during the procedure if current conditions suggest an increase or decrease is warranted (again with any of the aforementioned communication means for making such an adjustment).

As shown in FIG. 1, system 10 has injection-measuring device 4 connected to viscoelastic tubing 19. It should be noted that although injection-measuring device 4 is shown as being located in controller 12, it can be located anywhere along the viscoelastic tubing 19 or anywhere near the point where the viscoelastic materials enter into the eye. Also, injection measuring device 4 may utilize any means known by those skilled in art for measuring the volume and/or speed of fluids such as viscoelastic materials.

In such an embodiment, controller 12 may be programmed to compare the measured injection speed and/or volume and compare that information to desired levels of injection speed and/or volume and/or as a feedback means to maintain the desired flow speed and pressure levels dictated by the coordinating intraocular pressure monitoring system and processor which determine the needed one or more of flow, speed and pressure levels of the eye injected viscoelastic material relative to the eye's sensed intraocular pressure level. Based on such a comparison by the controller, there can be adjusted, for example, the volume and speed of the injection of viscoelastic materials into the eye, including turning the viscoelastic pump on and off or a reduction in pump output, to achieve the desired level(s). As shown in FIG. 1, the controller programming is provided with viscoelastic material flow characteristics as by way of injection measuring device 4, preferably together with an ambient temperature sensor input (e.g., any conventional temperature gauge that can provide means for inputting to the controller the present ambient temperature that is associated with viscoelastic material flow along viscoelastic tubing 19 with temperature sensor TS shown).

For example, injection measuring device 4 can be represented by any conventional flow meter suited for relatively viscous material (such as the Viscoat® and Healon® viscoelastic material referenced above) including a positive displacement flow meter that can be positioned, for example, along the feed line extending between the pump outlet and the viscous feed to the eye outlet (as at the injection-measuring device 4 (FIG. 1) or at the handpiece which is another suitable location for injection-measuring device 4) wherein the flow meter flow sensor can provide suitable real-time flow feedback to the controller and processor (e.g., the flow meter is suitable for monitoring both the flow and speed of the viscoelastic material flow at the desired location for monitoring, as in one that is close to the ultimate outlet to the eye (with the handpiece being well suited for this purpose as it provides a support base as well as close proximity to the viscous flow outlet to the eye)).

Examples of suitable positive flow meters include positive displacement flow meters which measure process fluid flow by precision-fitted rotors as flow measuring elements. Known and fixed volumes are displaced between the rotors. The rotation of the rotors are proportional to the volume of the fluid being displaced.

The number of rotations of the rotor is counted by an integral electronic pulse transmitter and converted to volume and flow rate.

The positive displacement rotor construction can be done in several ways as, for example:
Reciprocating piston meters that are of single or multiple-piston types.
Oval-gear meters that have two rotating, oval-shaped gears with synchronized, close fitting teeth. A fixed quantity of liquid passes through the meter for each revolution. Shaft rotation can be monitored to obtain specific flow rates.
Nutating disk meters have movable disks mounted on a concentric sphere located in spherical side-walled chambers. The pressure of the liquid passing through the measuring chamber causes the disk to rock in a circulating path without rotating about its own axis. It is the only moving part in the measuring chamber.
Rotary vane meters consist of equally divided, rotating impellers, of two or more compartments, inside the meter's housings. The impellers are in continuous contact with the casing. A fixed volume of liquid is swept to the meter's outlet from each compartment as the impeller rotates. The revolutions of the impeller are counted and registered in volumetric units.

Knowing the volumetric flow rate provides for versatility in determining various parameters that can be monitored with the processor for comparison with desired (target) levels as well as capped (maximum) levels that provide for limiting flow in those situations where there is concern that the flow rate or flow velocity has exceeded maximum levels (e.g., safety protocol maximum flow rate or velocity). For example, using the flow formula (1)

Q=V/t=A*v (1)

with Q being the volumetric flow rate; V being the volume of fluid; t being time; A the cross-sectional area of the fluid; and v the speed or velocity of that fluid there can be monitored flow characteristics in the system. Thus, for example, with FIG. 1 as an example, the injection measuring device 4 in the form, for example, of a positive displacement flow meter monitoring flow in feed line or viscoelastic materials tubing 19, provides for a calculation based on a known flow cross-sectional area such that the fluid area is equal to the cross-sectional area of the flow meter's predetermined area or that of conduit, as in A=πr².

The processor thus is provided with a current injection status data input from injection measuring device 4 using an above described conventional fluid flow sensor, as in one of the aforementioned volumetric flow rate sensors. With this information there can be maintained, lowered, or increased the flow output from the viscoelastic material pump through illustrated interface connection IL shown in FIG. 1. The lowering can be based on, for example, a start/stop pump motor technique or a variable rate motor can be utilized that increases/decreases flow rate via variation in motor (or actuator drive) output.

There can be monitored/adjusted the output level of the pump 14 to achieve a desired flow velocity and volumetric flow rate at the output end of the needle 26 (relative to the FIG. 1 embodiment). This line flow information can be coordinated by the controller relative to input user parameters and/or feedback from intraocular pressure monitor system 8, which input(s) are provided to controller 12. In this way, there can be coordinated the proper flow amount being fed into the eye based on the sensed intraocular pressure (or the flow sensor 4 can be utilized independently of the intraocular monitoring system 8 based on other inputs as in the aforementioned pre-set or real time operator inputs as to desired flow volume, speed, line pressure or any combination or sub-combination of these three parameters).

It should be understood that although system 10 shows viscoelastic material pump 14 integrated into controller 12, the present invention is not limited as such. It is contemplated that viscoelastic material pump 14 can be located external to controller 12. In such a case, viscoelastic material pump 14 can be controlled by controller 12 or a separate controller, wherein the separate controller can be a stand-alone device or the separate controller can be integrated into the viscoelastic pump or the handpiece 11. It is also contemplated that the viscoelastic material container 13 and the viscoelastic material pump 14 can be integrated into handpiece 11 while the viscoelastic material container 13 can also be removable therefrom for purposes of filling with viscoelastic materials or replacement. For ease of handling of the handpiece and/or for providing for sufficient viscoelastic material capacity, however, having the viscoelastic material source at least partially (e.g., a larger viscoelastic material source and a smaller handpiece viscoelastic material sub-container) removed from the handpiece is desirable. For example, having the pump feed viscoelastic material through a maintainer separate from the phaco tool (as shown in FIG. 2 and described in greater detail below) inclusive of feeding viscoelastic material through an independent eye incision location (i.e., an incision separate from the phaco tool with typically associated, same incision, irrigation feed and/or aspiration tube opening). The viscoelastic material (inclusive of different viscoelastic “materials”) container 13 can also be made of easily assembled components and materials such that it may be entirely disposable.

Thus, although system 10 has a single handpiece 11, wherein the functions of cutting, irrigation, ultrasonic emulsifying, aspiration, and injecting viscoelastic material are all integrated into one handpiece 11, the present invention is not limited as such. It is contemplated that such functions can be implemented through any number of handpieces or separate intraocular insertion devices (e.g., a conduit with injection cannula). For example, it is contemplated that one handpiece can be configured to provide the cutting, irrigating, emulsifying, and aspirating, and a second handpiece or intraocular insertion device can be configured to provide the injecting of viscoelastic material.

In other words, it is contemplated that the viscoelastic material can be dispensed into the eye without having to go through a handpiece at all. Instead, the viscoelastic material can be pumped through a tube such as, for example, a maintainer having a long braided tip inserted into an incision in the eye (e.g., an added second incision to a first incision receiving the phaco-tool).

Referring now to FIG. 2, there is shown an exemplary system 30 comprising means for dispensing or injecting viscoelastic material into an eye using a maintainer in accordance with the present invention. For simplicity, system 30 has a similar structure to system 10 shown in FIG. 1 and described, except for the means for dispensing or injecting viscoelastic material into the eye. In system 10 (FIG. 1), the means for injecting viscoelastic material into the eye comprised a processor 10 electrically connected to a viscoelastic pump 14, wherein the pump is connected to a viscoelastic material container 13 and a phacoemulsification handpiece 11, wherein upon activation of the pump by processor 5, viscoelastic material would be pumped from viscoelastic material container 13 to handpiece 11, which would inject the viscoelastic material through needle 26.

In contrast, the means for delivering/injecting viscoelastic material into the eye in system 30 comprises a processor 50 electrically connected to a viscoelastic material pump 35, wherein pump 35 is connected to a viscoelastic material container 42, and a maintainer 39, wherein maintainer 39 has a first end connected to a port on a controller 33 containing the pump 35 and processor 50, and a second end comprising a long braided tip 61 (shown inserted into the eye as in a common insert receiving the phaco-tool, or, more preferably, another incision location in the eye). As a result, when processor 50 activates pump 35, viscoelastic material from viscoelastic material container 42 are dispensed to maintainer 39 and injected into the eye through the long braided tip 61.

The following provides a more complete description of system 30. As shown in FIG. 2, system 30 comprises a controller 33, a handpiece 32, and a maintainer 39, wherein handpiece 32 is shown having a needle 34 and an aspiration tube 60, each inserted into an eye as in through a common, or more preferably, a different incision in which the maintainer 61 is inserted. Needle 34 (as a phaco-tool) includes an ultrasonically-driven horn and a needle tip configured to cut eye tissue, and may be of any conventional suitable design heretofore used in phacoemulsification handpieces. Similarly, as in conventional phacoemulsification handpieces, handpiece 32 is electrically connected (not shown) to controller 33 such that controller 33 can vary the ultrasonic power delivered by handset 32 through needle 34 for the emulsification of tissue in the eye. Thus, similar to conventional phacoemulsification hand pieces, the tip of needle 34, upon vibrating at ultrasonic frequencies, is capable of cutting or fragmenting eye tissue. Thus, needle 34 provides means for radiating ultrasonic energy into an eye in order to cut, fragment or emulsify tissue, depending on the particular surgical procedure being conducted.

Controller 33 comprises a processor 50 electrically connected to a computer-readable medium 51, an aspiration pump 38, a pressure detector 52, a valve 36, a viscoelastic material pump 35, and an electrical switch 70 on handpiece 32. Processor 50 is operable to call programs stored on computer-readable medium 51. The programs stored on computer-readable medium 51 include, but are not limited to, a program for controlling the operation of viscoelastic material pump 35, a program for controlling the operation of aspiration pump 38, a program for controlling the operation of pressure detector 52, a program for controlling the operation of valve 36, a program for controlling the ultrasonic power delivered by handpiece 32 to needle 34, and a program for processing an electrical signal from electrical switch 70 on handpiece 32. Processor 50 can call and execute these programs from computer-readable medium 51, as needed.

Viscoelastic material pump 35 is connected to a viscoelastic material container 42 through a tube 43 and to a maintainer 39. Viscoelastic material container 42 can be any type of container suitable for holding and dispensing viscoelastic materials. Viscoelastic container 42 can be refillable or removable such that replacement viscoelastic materials can be added to container 42 or a replacement container can be attached to tube 43, as needed. Further, viscoelastic container 42 is configured such that when attached to tube 43 and upon activation of viscoelastic pump 35, viscoelastic material in container 42 can be drawn through tube 43 by viscoelastic pump 35, and pumped to maintainer 39. Maintainer 39 has one end 62 connected to a port on controller 33, and a long braided tip 61 at the other end. Long braided tip 61 is shown inserted into the eye. Long braided tip 61 and end 62 of maintainer 39 are connected by a tube configured for carrying viscoelastic materials therethrough. The viscoelastic material container 42 can contain any type of viscoelastic materials including, for example, Viscoat® and Healon®.

Valve 36 is connected to an irrigation bag 48 through a tube 46 and to handpiece 32 through an irrigation tube 40. Irrigation bag 48 can contain any type of irrigation fluid including, for example, a saline solution. Irrigation bag 48 is removable such that a replacement bag can be attached to tube 46, as needed. Irrigation bag 48 is configured such that when attached to tube 46 and upon the opening of valve 36, the irrigation fluid in irrigation bag 48 can be fed through tube 40 to handpiece 32 and injected into the eye through needle 34.

Aspiration pump 38 is connected to handpiece 32 through aspiration tube 41, and to drain 37 through tube 45. Drain 37 can be any type of container for collecting aspirated fluids, materials and emulsified tissue from the eye. Upon activation of aspiration pump 38, fluids, materials and emulsified tissue in the eye can be drawn or vacuumed from the eye through sleeve 60 to aspiration tube 41 and fed to drain 37 through tube 45.

Pressure detector 52 is electrically connected through an electrical cable to an intraocular pressure-measuring device consisting of an intraocular pressure sensor 54 (shown inserted into the eye) and a transducer 53. The intraocular pressure sensor 54 is operable to measure the intraocular pressure of the eye and transducer 53 is operable to convert the measurement to an electric signal transmitted to pressure detector 52. The different types of devices and methods for measuring intraocular pressure using a sensor inserted into the eye are described above relative to the FIG. 1 system 10.

In operation, in accordance with the techniques disclosed herein, during surgery, viscoelastic materials are injected into the eye as a function of intraocular pressure. Processor 50 calls the program for monitoring the intraocular pressure form computer-readable medium 51. During execution of the program, processor 50 directs pressure detector 52 to utilize intraocular pressure sensor 54 and transducer 53 to obtain a measurement of the intraocular pressure of the eye. The measured intraocular pressure level is reported to processor 52, which compares the measured pressure level to a desired target pressure level. If the measured level is below the target level, processor 50 activates viscoelastic materials pump 35, unless viscoelastic material pump 35 is already activated. Upon and during activation, viscoelastic material pump 35 draws viscoelastic materials from container 42 and pumps the materials to maintainer 39 which thereby injects the viscoelastic material into the eye through the long braided tip 61. If the measured intraocular pressure level is at or above the target intraocular pressure level, the processor 50 deactivates the viscoelastic material pump 35, unless the pump is already deactivated. In similar fashion to the above described injection measuring device 4 in FIG. 1, injection measuring device 62 in FIG. 2 monitors one or more of the line flow, volume, speed of viscoelastic material being injected in the eye via maintainer 39.

Viscoelastic materials can also be injected on demand by the surgeon. In the event that the surgeon wants to inject additional viscoelastic material, even though the intraocular pressure may be at or above the target level, the surgeon can activate the viscoelastic pump by operating switch 70 on handpiece 32. Upon detecting that switch 25 has been manually switched to the on state, processor 50 will activate viscoelastic pump 35, thereby providing viscoelastic material into the eye through maintainer 39 in the same manner as described above. Upon detecting that switch 70 has been manually switched to the off state, processor 50 will deactivate viscoelastic pump 35, unless it is determined that the measured intraocular pressure is below the target pressure level which, in that case, processor 50 would not deactivate viscoelastic pump 35, or sends out an auditory signal or some other indication requiring confirmation of a desire for pump deactivation when the current pressure is deemed low.

It should be appreciated that an exemplary method in accordance with the present invention utilizes the herein described systems and includes in one instance the measuring of the intraocular pressure of the eye, comparing the measured intraocular pressure to a target pressure level and, if the measured intraocular pressure is below the target pressure level, automatically activating a pump for dispensing viscoelastic material into the eye, wherein the viscoelastic material can be injected through a handpiece, a maintainer or by any other means.

In addition, the present invention may also include the steps of: (1) making an incision into the eye and introducing a phacoemulsification needle into the incision for cutting, fragmenting, and/or emulsifying eye tissue using a handpiece; (2) making a second incision into the eye and introducing either or both of an irrigation tube or viscoelastic injector outlet into the second incision for irrigating fluid and cut, fragmented and/or emulsified eye tissue from the eye and/or for supplying protective and intraocular cavity shape forming viscoelastic material; (3) introducing irrigation fluid proximate the needle into the eye; (4) measuring the intraocular pressure of the eye; (5) comparing the measured intraocular pressure to a target pressure level; (5) automatically dispensing viscoelastic material into the eye when the measured intraocular pressure is below the target pressure level.

The step of dispensing the viscoelastic materials can comprise the steps of activating a pump when the measured intraocular pressure is below the target pressure level, and deactivating the pump when the measured intraocular pressure is at or above the target pressure level.

It should be appreciated that in an alternate embodiment of system 30, in accordance with the present invention, viscoelastic material pump 35 can be a stand-alone viscoelastic pump. In such an embodiment, viscoelastic materials pump 35 can be located external to controller 33 which would be operable to communicate with and/or control viscoelastic material pump 35 through any type of communication medium including, by way of example, a wireless interface, a communication cable, or a combination of such medium. In operation, through such communication medium, processor 50 can communicate with or control the activation and deactivation of viscoelastic materials pump 35 as described above.

In another embodiment, system 30 may also control the injection of viscoelastic material into the eye based on the injection speed and volume. That is, viscoelastic material can be injected into the eye as a function of intraocular pressure, injection speed, injection volume, or some combination thereof. As shown in FIG. 2, system 30 has injection-measuring device 63 connected to maintainer 39. It should be noted that although injection-measuring device 63 is shown as being located external to in controller 33, it can be located anywhere along the maintainer 39 or anywhere near the point where the viscoelastic materials enter the eye. Also, injection measuring device 63 may utilize any means known by those skilled in art for measuring the volume and speed of fluids such as viscoelastic materials, inclusive of those described above for injection measuring device 4 of the FIG. 1 embodiment. In such an embodiment, controller 33 may be programmed to compare the measured injection speed and/or volume and compare that information to desired levels of injection speed and/or volume; and, based on such a comparison, can adjust the volume and/or speed of the injection of viscoelastic material into the eye, including turning the viscoelastic pump on and off, to achieve the desired levels. This monitoring includes a simultaneous interfacing of signals received from the intraocular monitoring system 53 and injection measuring device 63 with suitable real-time adjustments to maintain a desired one or more of injection volume, speed of injection, and pressure; both in the line and the ultimate intraocular pressure, which can be preset prior to the procedure or at a time during the procedure, as upon a need seen by the operator.

FIG. 1A shows viscoelastic material injection system 10′ which is a similar system as that of system 10 in FIG. 1, but has an added, hand-less mechanical injection system 96 that comprises hand-less injection start triggering means 97, which in the illustrated embodiment is in the form of a foot pedal switch. Foot pedal switch 97 is in communication with the same switch junction 25 described above in FIG. 1 which provides for hand triggering of a switch to generate a signal transmission to processor 5 to direct processor 5 to activate viscoelastic pump 14. When the hand switch 25 is switched to the off state, a signal is transmitted to processor 5 to deactivate the viscoelastic pump 14. The foot pedal switch 97 operates in similar fashion to activate and deactivate the viscoelastic pump, and can replace the handpiece switch at 25 (or 32 of FIG. 2) or be a supplemental switch, where either a hand switch on the handpiece or the foot switch can be utilized. Thus, the foot pedal or both can be utilized for pump activation and deactivation. Also, the foot pedal trigger preferably is of a type wherein depression will activate the pump when the current pump setting is non-activation, and another depression (following a spring back resetting of the foot pedal) will place the pump in a non-activation state from a current pump activation mode. Further, the non-activation state associated with the foot pedal is preferably a “pause” mode for the motor and not a full shut down of the motor. In an alternate aspect of the invention, the hand switch can be used to put power to or block power to the motor (on/off of motor) and the foot pedal can be used to toggle between pump (motor) run and pump (motor) pause states.

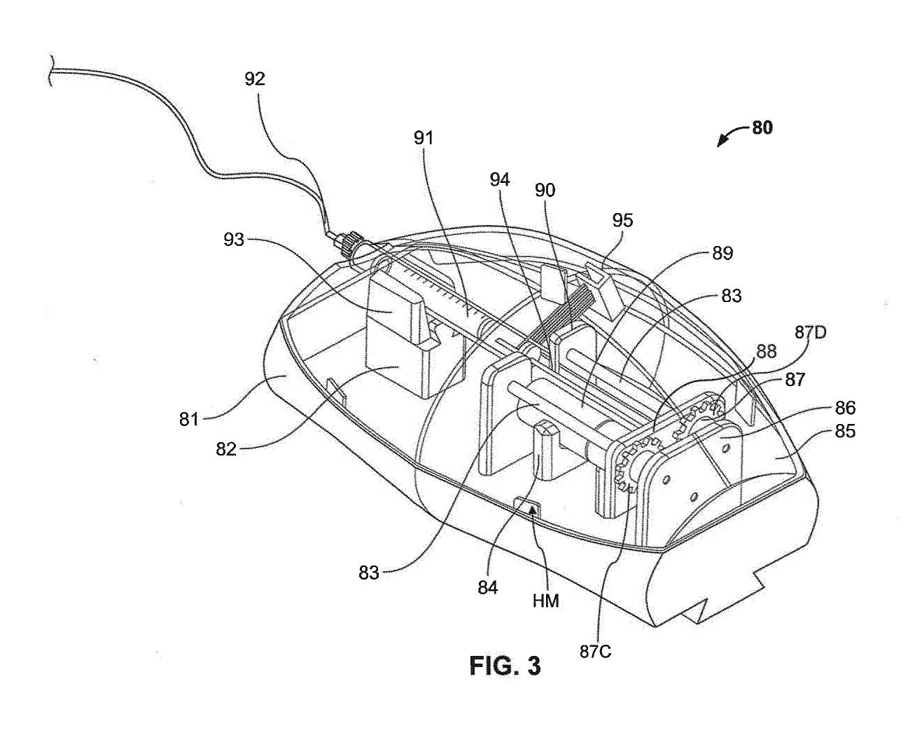

Referring now to FIG. 3, there is shown an exemplary embodiment of a stand-alone viscoelastic material(s) (material or materials at different times) pump 80 in accordance with the present invention. As shown, viscoelastic material pump 80 has a support base 81. Support base 81 is connected to and holds in place syringe guide holder 82, tab support 90, motor base 84, and gears support 86.

Syringe guide holder 82 has a top surface shaped to allow the attachment and removal of cylinder support 93, as desired. Cylinder support 93 has a curved top surface designed to support and hold in place syringe 91 when placed therein and to enable the removal of syringe 91 when desired. Syringe 91 has a cylindrical tube for containing a predetermined volume of viscoelastic material. Syringe 91 has a plunger 94 supported and held in place by tab support 90. Syringe 91 is connected to flexible cannula 92 and is operable to dispense viscoelastic material contained in its cylindrical tube through flexible cannula 92 when depressed. Flexible cannula 92 may be fed into the eye for dispensing the viscoelastic material into the eye during surgery such as in the context of maintainer 39 in FIG. 2.

Motor 89 is connected to and supported by motor base 84 which has a curved surface for holding motor 89 in place. Motor 89 is electrically connected to electrical interface 95 through which an external controller (not shown in FIG. 3) can control the power and speed of the motor, and turn motor 89 on and off. Motor 89 has a driving mechanism connected to gears 87 such that, when turned on, motor 89 can drive the gears 87 to rotate at a desired speed and power. Gears 87 are mechanically connected to threaded rods 83 which extend through fixed nuts (not shown) embedded in actuator 88. Actuator 88 moves along threaded rods 83 and has a surface that can push against plunger 94 when it moves towards syringe 91.

In operation, when motor 89 is turned on, its drive gear 87B drives gears 87C and 87D to rotate threaded rods 83A and 83B, respectively, to rotate along their axis in, for example, a counter-clockwise direction. The rotation of threaded rods 83 will mechanically interact with the nuts embedded in actuator 88 to drive actuator 88 to slide along the threaded rods and push on plunger 94. As plunger 94 is pushed, it will force syringe 91 to dispense viscoelastic material through flexible cannula 92 which may be fed into the eye during surgery. When motor 89 is turned off, it will stop driving gears 87 and thereby stop any dispensing of viscoelastic materials into the eye.

Thus, it can be appreciated that motor 89 can be any motor operable to generate the mechanical power needed to provide the functionality of viscoelastic material pump 80 described herein. For example, motor 89 can be a DCX16L 12V motor.

It can also be appreciated that although viscoelastic material pump 80 has been described in operation herein in connection with an external controller, viscoelastic material pump 80, in another embodiment, may contain its own controller that receives real-time measurements of intraocular pressure, and measurements of the speed and volume of viscoelastic material being injected into the eye during surgery. Based on any one or more of these measurements, the controller may turn viscoelastic material pump on and off to achieve the desired intraocular pressure, and speed and volume of viscoelastic material being injected.

Further, it should be appreciated that a viscoelastic material pump in accordance with the present invention is not limited to the embodiment shown and described for viscoelastic material pump 80. In an alternate embodiment, a viscoelastic material pump 80 may have multiple cylinder supports 93 for supporting multiple syringes 91, and a means for controlling and selecting which syringe 91 that actuator 88 engages to dispense viscoelastic materials into the eye.

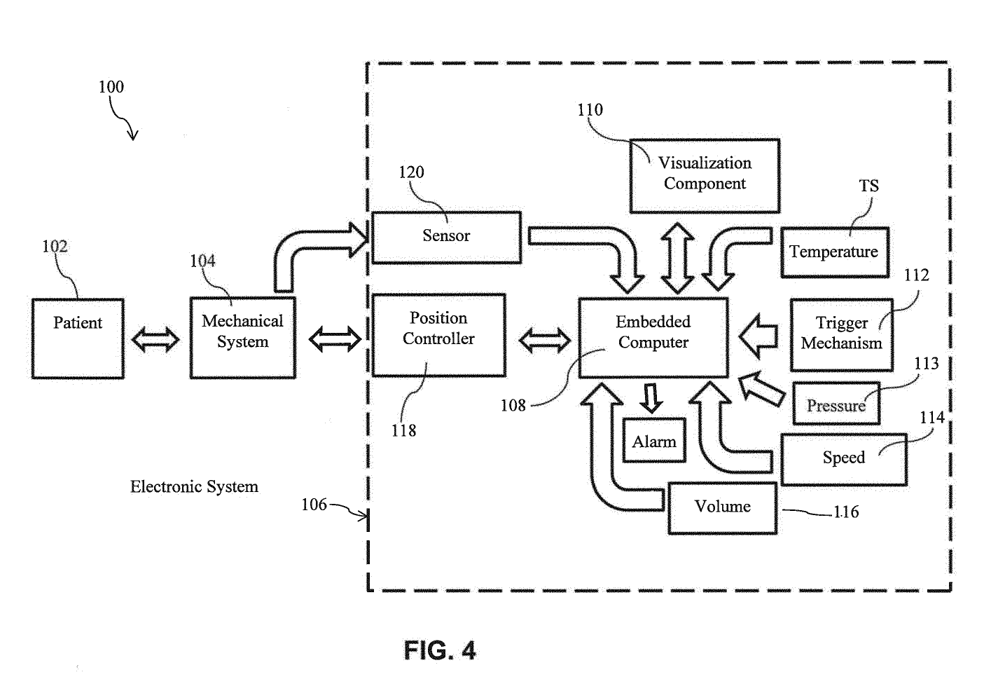

FIG. 4 shows a schematic view of an additional example of a viscoelastic fluid material injection system 100 well suited for delivering viscoelastic material to an eye during phacoemulsification cutting and/or fragmenting eye tissue (e.g., removal of a cloudy lens for replacement with a new lens).

As shown in FIG. 4, viscoelastic fluid material injection system 100 is arranged for achieving periodic injection of viscoelastic fluid in order to maintain the stability of the anterior chamber of the eye, thus preventing its collapse during a removal of eye material from the anterior chamber, while also serving to protect structures such as the cornea endothelium. That is, the injection system 100 helps in maintaining a desired eye shape despite ongoing suction out of eye material, while also providing the above described protective coating function.

The system 100 is well-suited to automatically and safely allow injection of the viscoelastic fluid according to a decision maker as in according to an operating physician's input and/or on the basis of a real time monitoring of system conditions and/or solely on preset conditions, with emphasis on ideal injection conditions, as in injection speed, injection volume and pressure of the fluid. For example, an operator or physician can input a desired set of settings considered best suited for the procedure to be performed and the system can be run with the goal of achieving the desired pre-set conditions during the procedure, with the preferable option of operator adjustment ability during the procedure (if deemed required) as by an interface (e.g., LED touch screen adjustment) that shows current settings of, for example, anterior eye chamber pressure, fluid speed, fluid pressure, fluid volume flow rate, and the fluid volume having been supplied since the trigger start (e.g., a depiction of the volume flow rate as well as total volume supplied as determined by, for instance, time of motor running multiplied by determined volume flow rate).

System 100 shown in FIG. 4 includes the following features shown as interfacing (as per the illustrated flow arrows (e.g., looped feedback programming directed between the processor and mechanical system, preferably inclusive of an electronic position controller associated with a DC motor for the pump as to provide during all times of operation knowledge of the current location and speed and direction of the driver and actuator providing the fluid injection release force to the viscoelastic material from the supply container)).

FIG. 4 shows schematically an interrelationship between an object receiving the viscoelastic fluid as represented by patient 102 such as one having cataract surgery, wherein the delivery of the viscoelastic fluid to maintain stability of the anterior chamber (despite ongoing irrigation with saline supply and suctioning of material out of the anterior chamber) and to protect corneal endothelium cells is achieved by providing a protective coating of the viscoelastic fluid.

Recipient or patient 102 is shown as interfacing with mechanical system 104 (which is representative of mechanical means for injection of the viscoelastic fluid to a recipient such as patient 102). As explained in greater detail below, mechanical system 104 is shown as including a housing structure designed to hold a multitude of individual independent containers containing viscoelastic fluid (the same type of viscoelastic fluid or two or more different types of viscoelastic fluid that may be better suited relative to a current environment or recipient 102 condition). For example, two, three, four, five or more independent containers each with the same or different viscoelastic fluid, such as three filled syringes of viscoelastic material of one or more types, such as the aforementioned VISCOAT® adhesive viscoelastic and/or HEALON® cohesive viscoelastic; each viscoelastic material type having particular advantages and disadvantages as described in the aforementioned and incorporated U.S. Pat. No. 5,358,473 and U.S. Pat. No. 6,254,587.

Accordingly, the mechanical system 104 of the referenced aspect of the invention provides for a high degree of potential versatility as with respect to, for example, accommodating of different initial or real time situations wherein one type of the viscoelastic material may be better than another for a given situation. For instance, an embodiment of mechanical system 104 features a base structure designed to house and adjust, with adjustment means, any one of a plurality of inserted containers (e.g., syringe containers) with a feed control system as in a combination linear screw type actuator powered by a rapid response driver (e.g., a DC motor) with an interface relationship with an electronic system to provide controlled viscoelastic fluid injection to recipient 102. Such adjustment can also be relied upon to lesson downtime and potential contamination with a switch out of a supply container (e.g., a plurality of containers having the same viscoelastic material and adjustment of a fuller container to replace a less full container of the same material either relative to an on-going surgery for a single patient, or for supplying viscoelastic material for a plurality of patients in series (without concern for container replacement) and yet with the ability to use commercially available syringe type supply containers of the viscoelastic material).

FIG. 4 provides an example of an electronic system 106 well suited for providing the above described controlled viscoelastic fluid injection to recipient 102. As shown in FIG. 4, electronic system 106 includes embedded computer 108 which represents the central processing system of the illustrated embodiment (having programming characteristics such as those earlier described for the first embodiment controller 12 inclusive of a processor 5 and its interfacing computer program; which, as earlier referenced, can take the form of a computer-readable storage medium).

As seen and FIG. 4, embedded computer 108 is preferably one with a central processing unit that interfaces well with the illustrated visualization component 110, such as a two-way interference relationship, as in one that features a touchscreen visualization component 110 (e.g., LCD touchscreen device).

Embedded controller 108 is also shown as interfacing with an operator trigger mechanism 112 as in of the type featured as the aforementioned elective switch 25 (associated with a phacoemulsification hand piece). More preferably, however, trigger mechanism 112 includes (either alone or in combination with the hand switch 25) foot pedal switch 97.

FIG. 4 also shows a temperature sensor TS providing a temperature value to the embedded computer in view of the known effect that temperature has on viscosity levels of viscoelastic materials such as those described herein (e.g., such temperature value sensing can be used to ensure fluid rate values are taken within a preset temperature range or known adjustments can be made in the viscoelastic flow monitoring calculations via computer 108).

As an additional aspect of the present invention, trigger mechanism 112 can also be represented (either in addition to, or as a replacement, of one or both of a foot switch and a handle switch) by a touch screen option in visualization component or visualization system 110. That is, trigger mechanism 112 can take on the form of a hand piece switch, a foot pedal switch, a voice command triggered switch, a visual display touch screen component, or any combination of these, which is representative of mechanical system operation triggering means as in a system for triggering mechanical system activation start and stop operation (e.g., pump activation and deactivation).

Embedded computer 108 is also shown as receiving input signals relative to pressure levels (input arrow 113), viscoelastic fluid velocity (input arrow 114) and viscoelastic fluid volume (input arrow 116). These inputs as to the viscoelastic fluid velocity and viscoelastic volume, in an aspect of the invention, represent real-time monitoring as to present fluid volume flow and flow line velocity that is determined in any of the manners described above (e.g., positive displacement monitoring). The real-time inputs 113, 114 in 116 are compared to the desired and/ or safe maximum pressure, flow velocity and volume settings that an operator can input using visualization component 110 or some other input means. With these inputs, the in line pressure level and/or in line volume and/or in line flow speed is monitored as described above and compared to preset parameters with one or more (e.g., all) of said values being displayed preferably together with the last operator set values for comparison purposes via visualization component 110.

If upon such a comparison it is determined that pre-set levels that have been set by the operator have been exceeded (preferably by a certain percentage over the preset value as in 10% to 20% or more over that value to provide for normal system fluctuations) or one or more threshold input levels suggests a dangerous condition approaching (e.g., intraocular pressure during surgery at a value above a predetermined cap value can be utilized as a shutdown pressure value (e.g., above 110 mmHg as in 120 mmHg). In any case a suitable alarm can be generated (e.g., sound, light, both sound and light, with or without automated remedial action as in system shut down or system adjustment as in lessening of the drive power used for pump operating. etc.). The alarm can also be a feature of visualization component 110. For example, if a first threshold is reached (as in 110 mmHg) an initial warning can be sent in which case an operator is put on notice, and if the pressure goes beyond that to a second threshold value (as in 115 mmHg) a pump shut-down or other flow blockage steps can be undertaken.

FIG. 4 further includes position controller 118 which provides a means for position adjustment of a viscoelastic fluid injection actuator as well as means for monitoring; at all times, and in real time, the relative location of the mechanical actuator (an example of a suitable actuator is pusher 206 described below). Additionally, the position controller 118 provides for the supply of additional system information as in the real-time variables of operation of the motor (driving a mechanical fluid flow actuator) inclusive of torque, operating speed and nominal velocity of the driver as in a DC motor (as in the aforementioned DCX16L motor having a reducer of 16:1, and an associated position encoder and amplifier). Excessive values of these types (e.g., excessive torque) can also be utilized to detect system issues and, if detected, can trigger alarm and/or system shutdown (or a flow stopping technique).

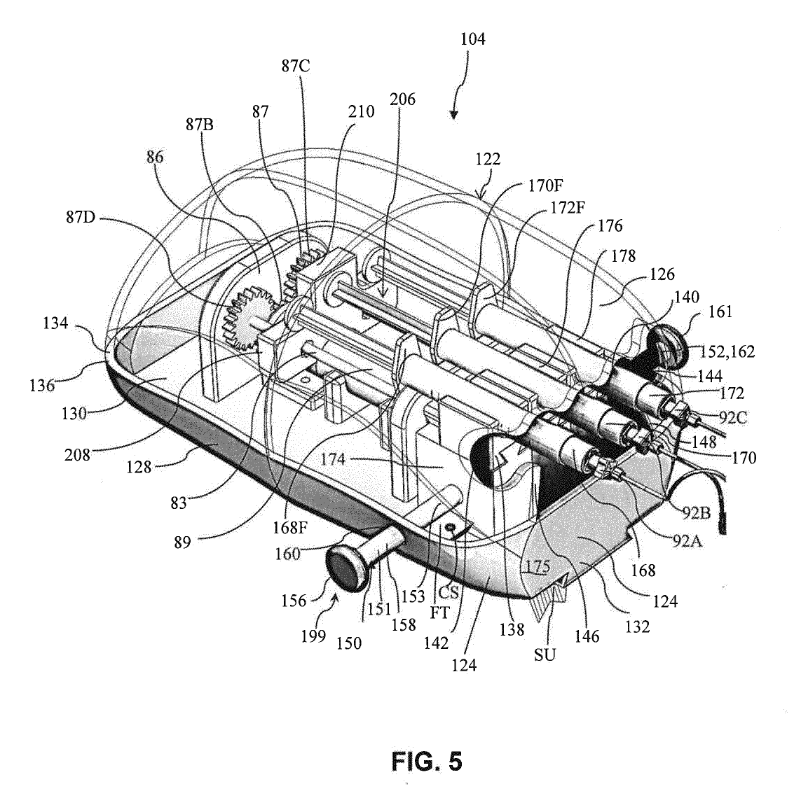

As explained in greater detail below, electronic system 106 further includes sensor system 120 which receives data input from mechanical system 104 which enables for adjustment of mechanical system 104 to set or reset the injection system into an initial feed orientation. Sensor system 120 preferably also includes, for the adjustable multi-container system shown in FIG. 5, a side-to-side container support position sensor as in one or more contact sensors provided in the container support path to provide information as to which container is or has been aligned with actuator 206 in the below described FIG. 5 embodiment. This side-to-side position information is helpful in the virtual display of each of the containers fill level.

Embedded computer 108 further features a backup battery system (not shown) to retain the last real time position status in the event of an electricity disruption which avoids having to recalibrate in such situations. Alternatively, the entire system can be battery based to provide greater mobility and positioning options.

As noted above, sensor system 120 further provides for using the readings from position controller 118 to evaluate, as a safety mechanism relative to an on-going procedure. For example, the maximum fluid supply pressure in conjunction with an analysis of the torque output of the motor. That is, if the torque value exceeds a certain value that is indicative of a blockage or other obstruction relative to fluid flow in the system as the motor moves into a higher torque mode due to such an obstruction.

FIG. 5 illustrates an additional example of mechanical system 104 featured in a viscoelastic fluid injection system 100 under the present invention (which thus provides viscoelastic pumping means like pumps 14 and 35 featured in FIGS. 1 and 2). As shown in FIG. 5, mechanical system 104 shares some similarities with the standalone viscoelastic materials pump of FIG. 3, but also includes some differences that become apparent in the discussion below. Mechanical system 104 of FIG. 5 is shown in the form of a pumping system that includes housing 122 featuring base housing body 124 together with housing cover 126. As seen, base housing body 124 has side walls 128 and floor 130 which together define an interior reception cavity for various components of mechanical system 104. Floor 130 is shown with an elongated keel 132 on its underside having a dovetail configuration which provides for a slide or insertion and position retention as relative to a support SU (schematically depicted) near the location of operation (e.g., a support table) or a pushcart with roller and brake to provide greater flexibility in positioning the viscoelastic injection system of the present invention in various locations close enough for the injection system to reach an intended fill area as in the eye chamber, but out of the way of the surgeon, etc.

Cover 126 is also shown as being transparent or translucent such that components inside the house can be visualized. A suitable plastic material such as clear or translucent plastic material is utilized that can be readily molded and subject to sterilization techniques (as in autoclave heating without plastic melting).

Cover 126 is further shown in FIG. 5 as having a peripheral edge 134 that rests on peripheral edge 136 of base housing body 124. The interrelationship between cover 126 and base housing body 124 is such that cover 126 stays in position until it is desired to access the interior of base 126. For example, an overhanging flange relationship to prevent slide off and/or the inclusion of hinge means (e.g., see HM in FIG. 3). In addition, FIG. 5 shows housing 126 having an aperture 138 defined by castellated upper edging 140, that borders at its ends with outer side recess cavities 142, 144 that extend inwardly to side edges 146, 148 defining the opening provided in the lower part of aperture 138 (an area below the cantilevered extending viscoelastic material containers).

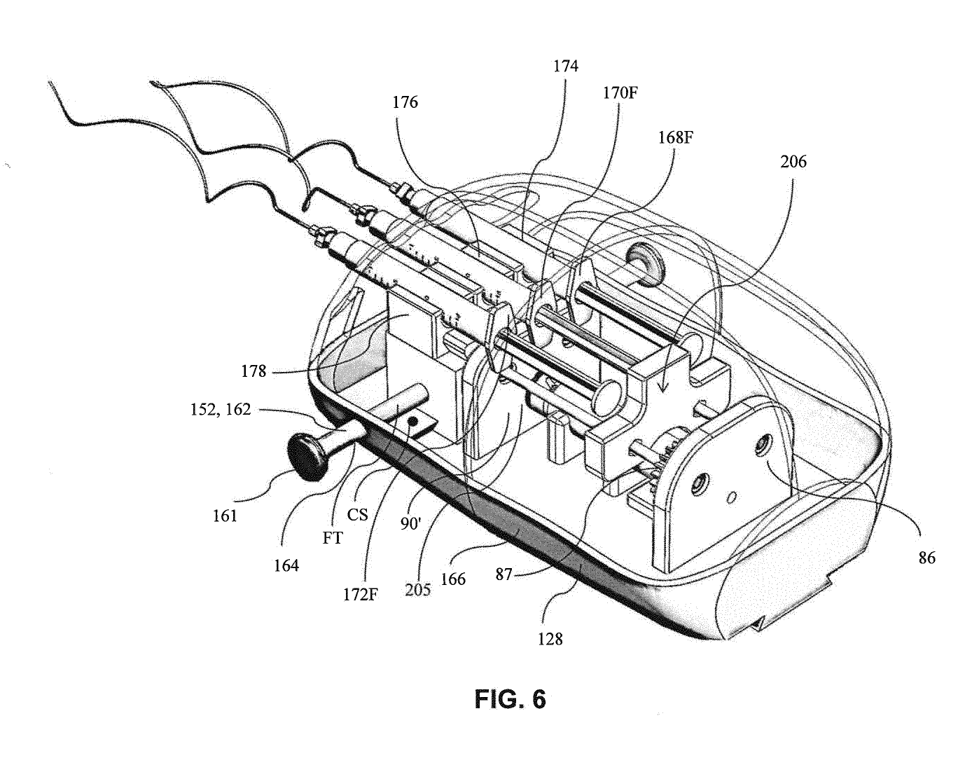

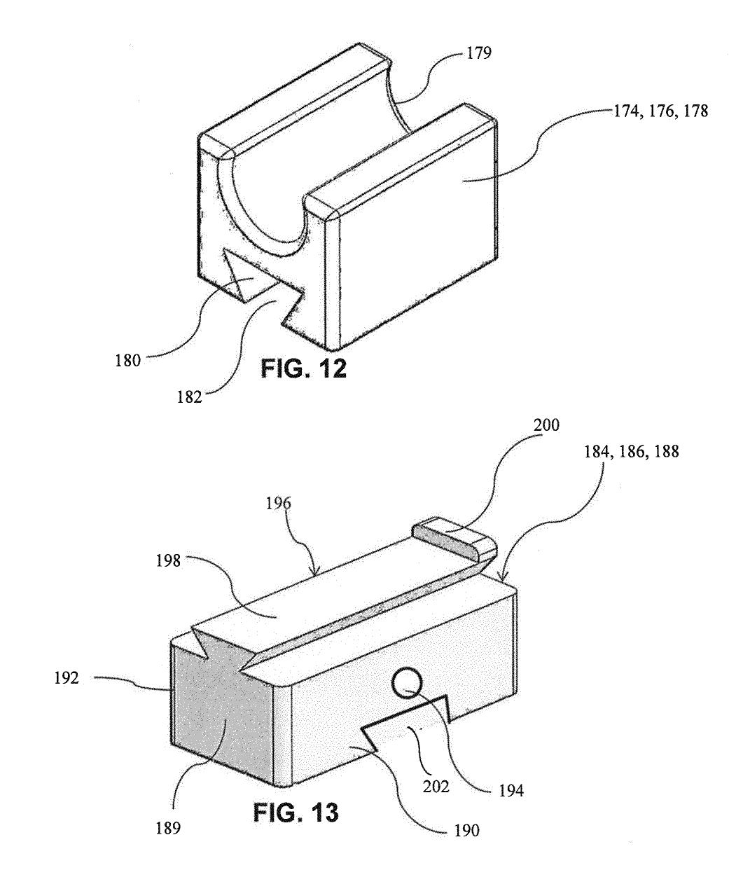

FIG. 5 illustrates first and second (e.g., left and right) exposed ends 151 and 152 of push-pull member 150. Exposed end 151 of the rod shaped push-pull member 150 features a free end grasp handle 156 and an attached shaft section 158 shown extending through slide hole 160 formed in sidewall 128 of base housing body 124. Similarly, the second exposed shaft end 152 of push-pull member 150 has a free end grasp handle 161 from which extends shaft section 162 extending through a similar slide hole (164, FIG. 6) in opposite side wall section 166 of base housing body 124. Push-pull member 150 thus preferably features single shaft unit 153 comprised of the aforementioned exposed shaft sections 158,162 having opposite grasping handles 156, 161 and with shaft unit 153 extending through both side wall sections of the base housing body 124 as well as fully through each of three guide holders 184, 186 and 188 (FIG. 13, described below) provided on floor track FT (having a dovetail cross-section 204) within the housing and also supporting a contact sensor CS (e.g., photodiode or electrical contact, etc.) with suitable wire feed to the controller (not shown).

Thus, the present invention includes an aspect wherein there is provided a viscoelastic material support assembly that includes a container retention support structure that is formed by the combination of the aforementioned set of cradles and underlying set of support blocks. The viscoelastic material support assembly also includes an adjustment mechanism (or means for adjustment 199) that features, in the embodiment shown push-pull member 150 having the aforementioned shaft unit 153 and opposite end grasp handles 156 and 161.

The viscoelastic fluid material containers 168, 170 and 172 are also shown as extending within housing 122 as well as through aperture 138 formed in cover 126 and out away from section 175 of base housing body 124. Castellated edging 140 is also shown as being designed to have edging surfaces that conform, at least in part, to a curvature in the exterior section of containers 168, 170 and 172, and is also preferably designed to at least partially extend below an upper level of the containers as to provide a degree of position retention or at least means for limiting relative movement freedom within the housing (i.e., castellated edging 140 is shown to present a movement limiting means both relative to upward and side-to-side container movement, while still providing for receipt of various diameter containers as in those with aforementioned VISCOAT® and HEALON® visco materials).

Further, the partially circular, outer side recess cavities 142, 144, positioned to opposite sides of the set of multiple containers, provide easy access for finger assertion and cover lifting (or dropping). For example, although hinging HM (FIG. 3) is shown as provided along the long sides of the housing base body, in an alternate embodiment (not shown), hinging is provided on the shorter side of base housing body 124 with outer side recess cavities 142, 144 proving an easy finger grasp location for hinged lift up of cover 126 in either hinge arrangement.

FIG. 5 still further shows each of the viscoelastic containers 168, 170 and 172 to be received (e.g., in a friction “snap-in” fit) within respective cylinder (container) supports or cradles 174, 176, 178. With such an embodiment, the underlying reception of the containers (designed to capture in position a viscoelastic container) renders, the above noted castellated opening 140 in the cover, a secondary confinement means in the event the cradle retention comes loose.

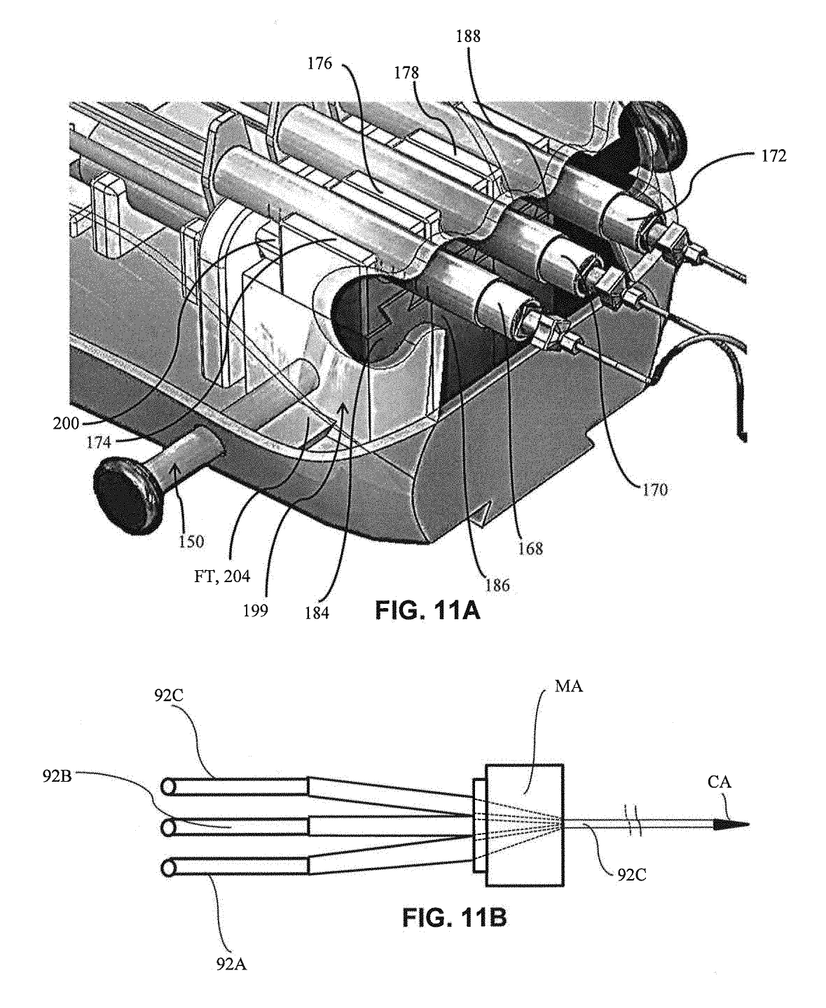

In a mode of the present invention, each cradle has a universal design or a common construction. Under this universal concept, the FIG. 12 illustration is representative of an embodiment of each of cradles 174, 176 and 178. As seen by FIGS. 11A and 12, each cradle has an upper side with a container reception 179 recess (shown in this embodiment as semi-cylindrical recess) and on its lower slide a slide connector segment 180, which in the illustrated embodiment is represented by a female dovetail shaped slide recess 182 (FIG. 12) for slide track attachment to the top of an underlying support block such as support block 184 shown in FIG. 13.

That is, as further shown in FIGS. 5, 11A, 12 and 13, provided below each of cradles 174, 176 and 178, there is located a respective one of guide holders or support blocks 184, 186 and 188. As with the cradles, in a mode of the present invention, each of the support blocks 184, 186, 188 is preferably universal, or has a common configuration. As shown in FIG. 13, each guide holder has an enlarged main body base 189; having, in the illustrated embodiment, a rectangular shape with a set of parallel long sides 190, 192 into which extends aperture 194 (shown as centrally positioned in FIG. 13). Aperture 194 is preferably a through-hole (e.g., providing for the noted universal nature, although other arrangements are also featured under the present invention as in threaded apertures to each side and intermediate threaded stub shaft to provide for side-to-side slide adjustment upon push-pull member 150 manipulation of all aligned support blocks). Aperture 194 thus provides a reception hole for receipt of the shaft unit 153 of the rod shaped push-pull member 150. In the illustrated example of the invention, the shaft unit 153 is appropriately fixed (e.g., adhered or threaded on) in position within the three through-holes of support blocks 184, 186 and 188 such that the entire assembly (push-pull member and the three support blocks can slide side to side as a single unit). Push-pull member 150, with its interior shaft unit 153, and end handles 156 and 161 thus represents a non-automated hand manipulated version of adjustment means 199 for adjusting the position of the container retainer support structure (combined cradle and guide holder sets).

FIG. 13 further shows each guide holder or support block 184, 186, 188 having an upper slide engagement member 196 featuring an elongated rail 198 and a catch 200. Each rail 198 is provided with a dove-tail cross-section for slide reception within the corresponding dove-tail recess 182 provided at the base of each of the cradles. An alternate male-female locking means, that preferably provides for ready slide off and re-insertion, can also be implemented under the present invention inclusive of a reverse male/female relationship relative to the vertically stacked cradle and guide holder and the respective dove-tail configured receptors. Additionally, catch 200 is shown provided on the more internally positioned end of rail 198 such that, upon opening the cover, a desired cradle can be switched out with ease for insertion of a similar sized container (or a different sized container can be inserted into a different (conforming to new sized container) cradle having the same dove-tail connection arrangement as described above). Alternate blocking means (other than the described abutment contact, but one that preferably allows slide freedom in one direction provided as by catch 200) are featured in other aspects of the invention; as in a catch arrangement with a release tab or similar releasable fixation arrangements (not shown).

As further shown in FIG. 13, each guide holder 184, 186 and 188 also is shown as including a lower slide cavity 202 that is arranged as to extend parallel with the axis of through-hole aperture 194 provided in each of the guide holders and thus perpendicular to the direction of elongation of rail 198. A female connection arrangement component at lower slide cavity or recess 202 is shown in FIG. 13, which recess 202 is also shown as having a dovetail configuration (providing means for retained sliding, in that the guide holder can slide on a corresponding underlying track FT in the housing (male component of the means for retained sliding) while not lifting up).

FIG. 11A illustrates (partially) such a track “FT” (e.g., dovetail cross-sectional track 204 which represents one difference relative to the embodiment of FIG. 3 which is free of such a track). Track 204 is shown in FIG. 11A as extending parallel with the extension direction of the rod shaped push-pull member 150 and extends from one side wall 128 to the other side wall 128 and is fixedly secured to the floor of the base (e.g., after each of the three guide holders are slid into position on the track). An example of floor track FT to base housing body 124 includes forming alignment pins extending up from the floor of the housing for receipt within complementary pin recesses (not shown) at the base of the attaching floor track FT, whereupon after slide on insertion of the set of guide holders the pin/pin recess combination can be fixed as by an adhesive connection.

Under the arrangement shown in FIG. 11A, an operator is free to push or pull on push-member 150 which implements a side to side sliding reaction in the set of guide holders 184, 186 and 188, cradles 174, 176 and 178 (when slid into position on a respective guide holder) as well as containers 168, 170 and 172 (when slid into position on a respective cradle).

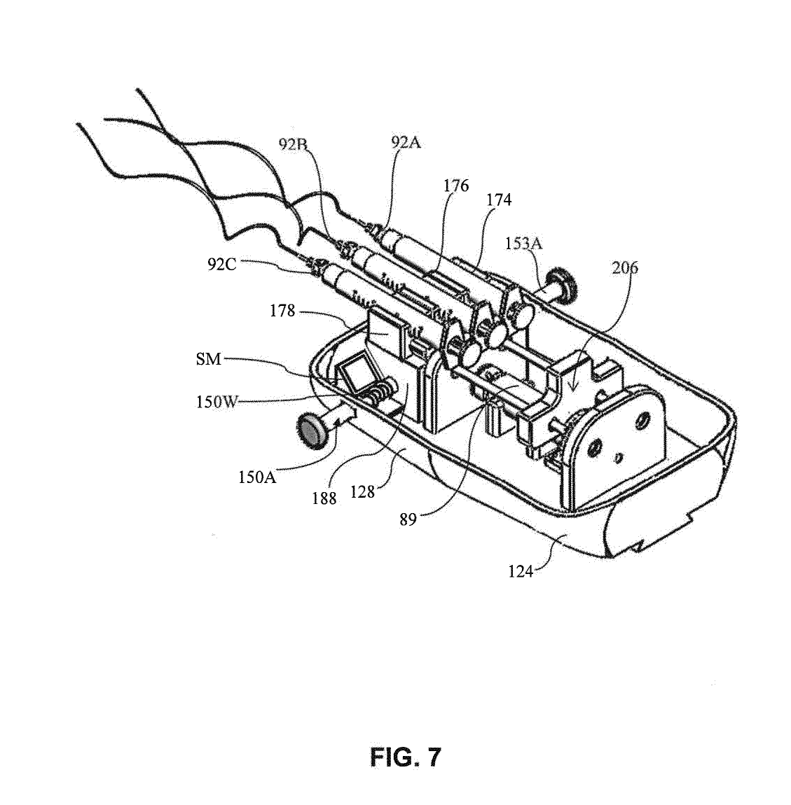

Further, rather than the manual version of the container side-to-side adjustment means 199 represented by the manual push-pull member 150, which works in association with the container retention supporting structure (represented by the sets of guide holder and cradles), alternate side-to-side adjustment means are considered under aspects of the invention, inclusive of automated versions (as in a pulley attached to the guide holders and a drive/driven spindle set mounted in the housing), and which automated version of the adjustment means 199 is controlled and activated by, for example, the aforementioned controller 12 or embedded computer 108. Such activation is inclusive of operator triggered activation as in a touch screen signal generation relative to visualization system 110 shown in FIG. 4 or some alternate triggering mechanism inclusive of a phaco tool handle switch setting. FIG. 7 provides a schematic illustration of one example of an automated container side-to-side adjustment means 199 having a push-pull member that features a worm gear 150W and sub-motor SM combination 150A that can drive push-pull rod 153A in either direction depending upon controller direction and to achieve the container repositioning described above and below (preferably with suitable left and right position contact switches CS (see FIG. 5)). To provide a more compact system base block 188 is shown with a notched recess to receive a portion of the housing of sub-motor SM, or alternatively, the exposed shaft portions on opposite sides (one of which has the worm gear threading) can be elongated for further side-to-side shifting clearance.

In an alternate aspect of the invention there is featured a monitoring system for monitoring the amount of viscoelastic system remaining; which, as described above can be achieved by a controlled monitoring of the drive motor's positioning of the actuator (e.g., the component pushing the syringe plungers) as wherein the amount of longitudinal actuator adjustment (e.g., plunger push in) is monitored and the controlled system adjusts the relative position when a low setting is sensed. Additional or alternate monitoring means includes a visual based monitoring system inclusive of one mounted in the housing that can monitor for a visual distinction between a fluid present and a fluid not present in a respective one of the multiple containers. The aforementioned side-to-side (left-right) contact switches CS also provide for logic monitoring of which container has been shifted into position for dispensing.

A comparison of FIGS. 3 and 5 shows both similarities and differences between the housing and contents of the housing in each. In view of the similarities between the two systems, the discussion below focuses on the differences, including the above described multi-container supporting side-to-side adjustment (slide) means 199 not featured in the FIG. 3 illustrated embodiment.

An additional difference, brought about by the multi-container capability of the FIG. 5 system, includes T-shaped actuator or activation block 206, instead of the rectangular (non-T-shaped) actuator 88 featured in FIG. 3. That is, while the presence of a DC motor and associated transmission (shown as including the gearing set 87 and a rotating linear drive assembly with threaded rods) designed to longitudinally move the actuator (having its own threads as in threaded nuts inserted into the main body 208 of the activation block) is the same in each system, the actuator or activation block 206 of the FIG. 5 illustrated arrangement features an underlying body section 208 (supporting the threaded nuts as in the earlier FIG. 3 embodiment), and further includes a centrally positioned and upward extending pusher section 210. The side-to-side width of upward extending section 210 (or activation block section or pusher section) is designed such that it avoids contact with each cylinder(s) not centrally positioned following adjustment of the containers by side-to-side adjustment means 199.

Thus, in the FIG. 5 illustration, side-to-side adjustment means 199 is shown in FIG. 5 as being set in a centralized position, which places central container (syringe) 170 in alignment with upward (centralized) activation block (or pusher) section 210. Upon motor 89 being placed in drive mode by the processor, and the activation block moving transmission placed in motion, syringe plunger (for container 170) is pushed toward the container outlet for injecting viscoelastic fluid flow to the eye. As noted above, the adjustment of activation block 206 on the threaded rods 83 is controlled with real time relative positioning monitoring such that when activation block 206 reaches the end (or near the end as in at least 90% of its full travel capability) of its forward travel, it can be automatically returned to the set position by the above described real time position monitoring means featuring the DC motor and encoder and controller coordination together with updating the system to note that container is now in an “empty” state. That is, upon driver (e.g., DC motor) 89 being activated by a controller signal, it initiates movement of the transmission (e.g., the gearing set 87, and linear screw arrangement which receives the drive force from the motor and translates it into actuator 206 adjustment) there is implemented a corresponding longitudinal movement in the container 170 plunger leading to viscoelastic material injection toward the eye.

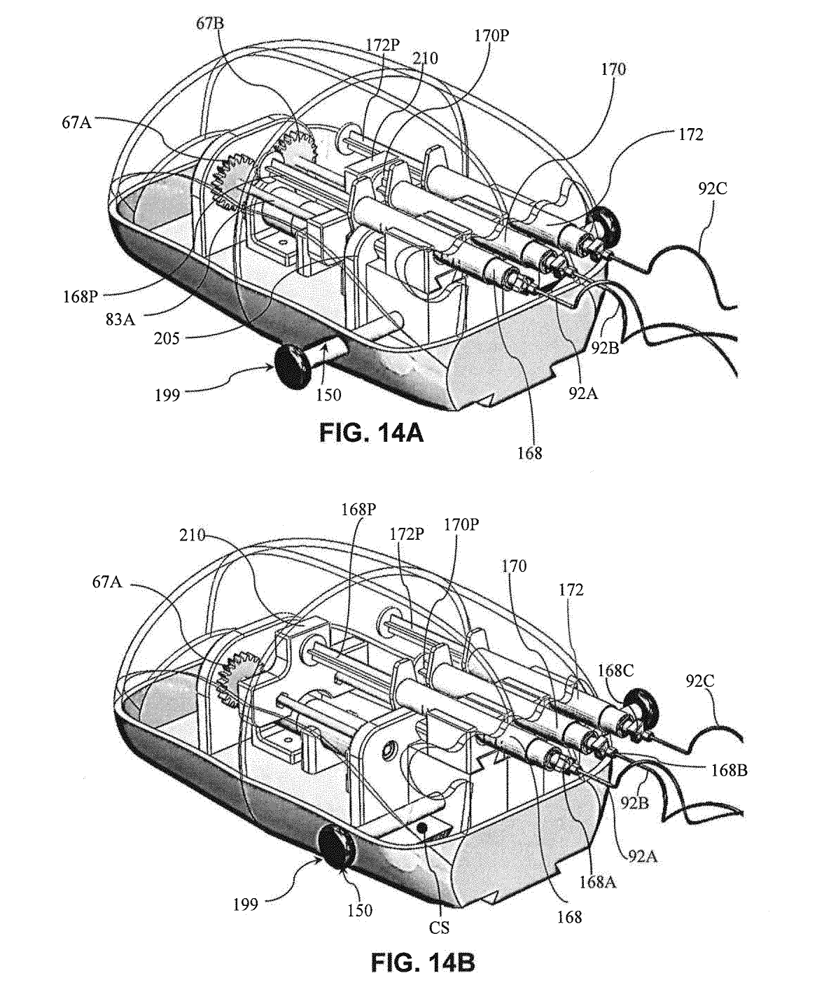

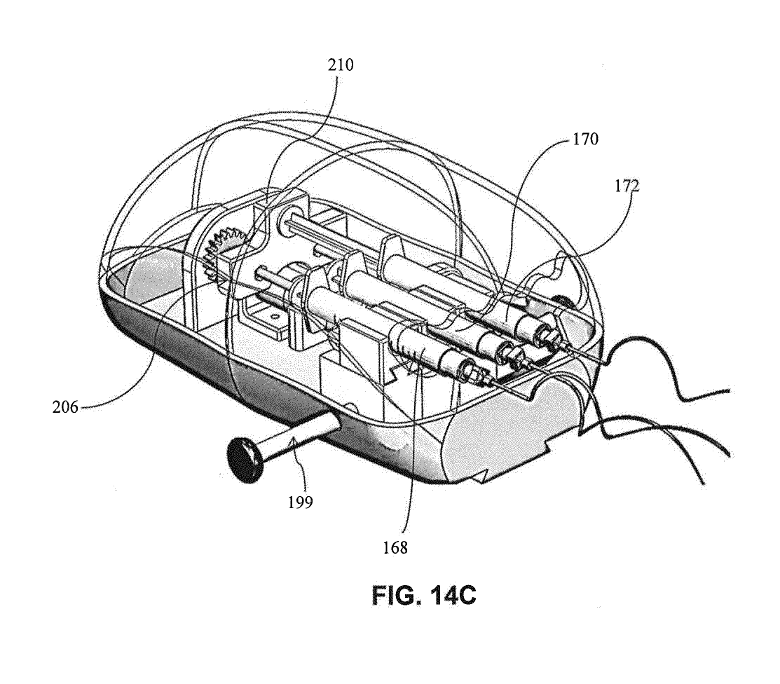

An illustration of activation block 206 having been driven by the controlled motor to push the plunger 170P to its full reception state within the cylinder portion of the corresponding syringe container is seen in FIG. 14A. As further seen in FIG. 14A, the relative width of upward extending section 210 is clear of the remaining two plungers 168P and 172P.

Upon longitudinal retraction of activation block 206 to the same position represented by the start position shown in FIG. 5 (wherein the pusher is clear of all plungers regardless of fill level of the containers), the side-to-side means 199 can be activated by push-rod adjustment (or other adjustment means such as the above described automated adjustment means 150A) until a desired one of containers 168 and 172 is aligned with the upward extending (central shown) pusher section 210 of activation block 206. FIG. 14B illustrates such an adjustment wherein the previously emptied container 170 is adjusted (to the right in FIG. 14B) such that the non-empty viscoelastic material container 168 is moved into alignment with pusher 210. In this way upon a signal received from the controller, as in upon an actuation triggering signal generated upon foot switch 97 (FIG. 3B) activation, the motor, and associated transmission actuator, leads to actuator linear adjustment and associated plunger depression within the horizontal syringe cylinder 168 and viscoelastic material flow through flexible conduit 92A.

The controlled motor driving of activation block 206 to the new container emptying mode is then carried out while the relative real time positioning is monitored. Upon that container reaching a similar empty state (i.e., container 168 reaching a similar empty state as the empty state represented by the earlier emptied container 170 in FIG. 14A), actuator (activation block) 206 can be automatically retracted to the re-start position, whereupon another container can be shifted into position (with the position shift detected by the controller) to be aligned with extending section or pusher 210, whereupon the sequence can be repeated. Such a further repeated adjustment to switch out an emptied container with a non-emptied container is illustrated in FIG. 14C. That is, following actuator 206 retracting and automated or manually implemented adjustment means 199 manipulation, there is achieved a side slide shift that adjusts the last remaining full container 172 from its far right position to the position shown in FIG. 14C, wherein container 172 is aligned with the central pusher section 210 of activation block or actuator 206. In this way, upon driver “on” triggering the material in the last remaining non-emptied container can be ejected out through flexible conduit 92C.

With the availability of multiple containers, as in those featured in the FIG. 5 system, there can be ensured sufficient viscoelastic material even for an extended operation and/or in situations where the containers utilized were not sufficiently full to start. Further, an appropriate warning signal (e.g., through interface screen 110) when the last container is approaching an empty state such that suitable steps can be taken for replacement (including a replacement of each of the now emptied multiple containers).

In other words, with this adjustment capability in the side-to-side adjustment means there is provided freedom to adjust one or more used containers out of the way such that a new or fuller container can be repositioned for activation by the actuator 206. There can also be provided for container adjustments during an operation wherein a container of different material is adjusted into position to replace another container earlier relied upon (e.g., where a more viscous material can be replaced with a less viscous material and vice versa to better suit current operation conditions).

FIG. 14A also shows retainer wall 205 as in abutment with the finger flanges of the respective containers 168, 170 and 172 which prevents longitudinal siding of the containers at a time when the actuator 206 is moving to compress the telescoping plunger extending in the fluid holding container of that syringe being emptied. Alternative retention means are also featured as in reception slots provided in holsters (not shown) supported on the same retainer wall 205 (this provides the added feature of an anti-rotation security relative to the syringe cylinders and retainer wall 205).

FIG. 6 shows an alternate perspective viewpoint from that of FIG. 5, and with the containers 168, 170, 172 each in a full state with actuator 206 in an initial contact state with respect to central container plunger 170P. Each of FIGS. 5 and 6 show container flanges (syringe finger flanges) 168F, 170F, 172F in abutment with tab support or retainer wall 205 (shown as similar to that of FIG. 3 embodiment but without the central notch shown in FIG. 3).

In other words, the coplanar relationship between flange tabs 168F, 170F and 172F and the abutting sidewall of retainer wall 205 is shown in FIGS. 6 and 14A provides for retention of the cylinder portion of the respective container as its plunger is subject to forward advancement by pusher 206. The co-planner contact between flange tabs (e.g., 170F) and tab retainer wall 205 allows for retainment of the syringe container cylinder as the plunger is advanced in controlled fashion by actuator 206 with upstream motor and motor transmission action while under the control of the controller (as in controller 12 described above in the FIG. 3 embodiment with associated sensing means and programmer such as programmer 5 in FIG. 3 and similar processing mean relative to embedded computer 108).

FIG. 7 shows an alternative perspective viewpoint from that of FIG. 5, but with the housing cover 126 removed (and with each of containers 168, 170, 172 having been emptied, and with actuator 206 in a retracted state (as to facilitate replacement of the empty containers)). That is, an operator can readily remove the empty container set 168, 170, 172 from their respective cradles 174, 176 and 178, whereupon a full set (e.g., three spots shown) of full containers can be newly inserted. FIG. 7 also features an automated adjustment means 199 using worm gear system 150A, per the discussion above.

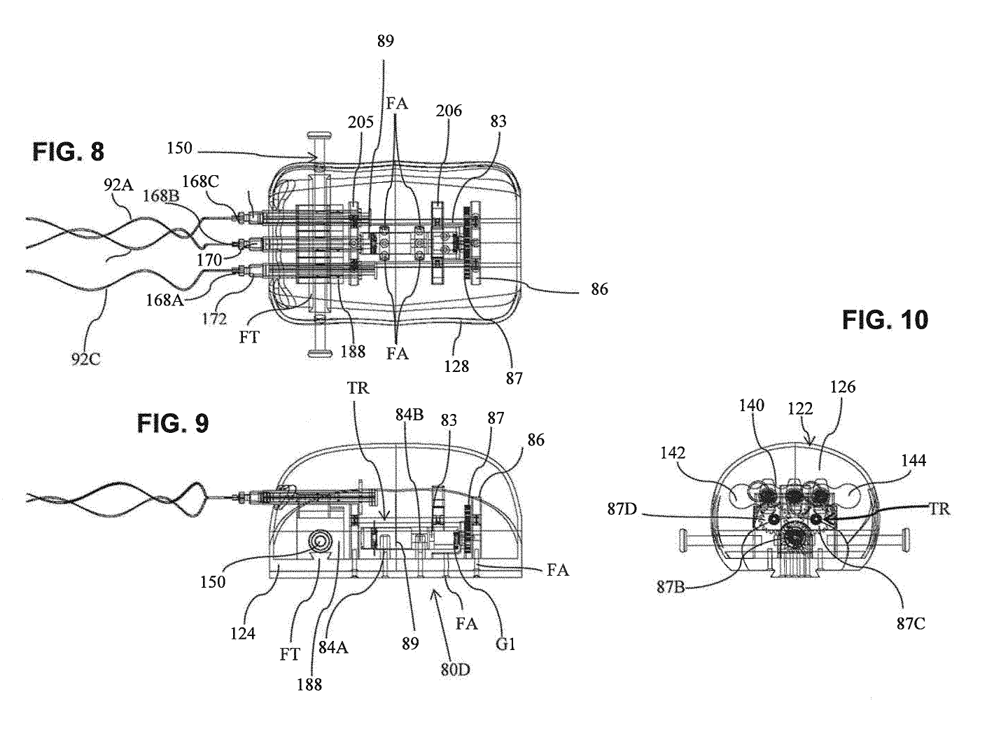

FIG. 8 shows a top plan view of the containers emptied embodiment of FIG. 7, and with push-pull member 150 in its central (neutral) setting. As further seen in FIG. 8, floor track FT (dovetail configuration floor track 204) runs nearly to the interior surfaces of the sidewalls 128 of body housing base 124. As further seen from FIG. 8, sliding guide holders or support blocks 184, 186 and 188 (see FIG. 13 for a universal depiction of the same) are dimensioned to ride along in side slide fashion on floor track FT. As further seen in FIGS. 8 to 10, motor mounts 84A and 84B are fixedly attached to motor 89 by way of fasteners “FA” extending up through the housing base floor into threaded reception regions in mounts 84A and 84B or by an adhesive pin connection.

FIG. 9 further shows additional fasteners FA extending up from (through) the floor of the housing base and holding in position the rigid components as in gear support 86.

FIG. 10 shows a front view of housing 122 wherein there can be seen opposite finger recesses 142 and 144 formed in housing cover 126 as well as the castellation recess edging 140 which extends above the respective containers 168, 170 and 172. FIGS. 9 and 10 also shows the transmission TR for driving the container plungers, inclusive of gear set 87 receiving a driving force from the motor output gear 87B (part of the driver motor 89 featured in this embodiment) mounted on the output shaft of the motor 89 as well as the drive gears 87C and 87D that are rotatingly mounted on gear support 86 and rotationally fixed to linear drive threaded rods 83. In this way, upon rotation of gears 87B and 87C of gear set 87, the respective threaded rods 83A and 83B rotate causing actuator 206 to ride forward (toward the container ends having flexible cannula tubes 92A, 92B and 92C having threaded connectors 168A, 168B and 168C).

FIG. 11B shows an arrangement that avoids having to remove a viscoelastic fluid injector needle from the eye upon a container emptying and allows for a switched over container to feed to a common eye chamber cannula CA (e.g., braided tip 61 of maintainer 39) previously inserted into the eye. That is, as shown in FIG. 11B each of the viscoelastic fluid injection lines 92A, 92B, 92C leaving the respective containers 168, 170 and 172 are secured to manifold MA which channels the viscoelastic material to a single outlet conduit 92C having it its free end a suitable cannula CA for insertion into a chamber of an eye receiving viscoelastic material (e.g., the injection end of maintainer 39). This manifold arrangement can also be conveniently located within handpiece 11 although alternate locations (both upstream and downstream of the handpiece) are contemplated.

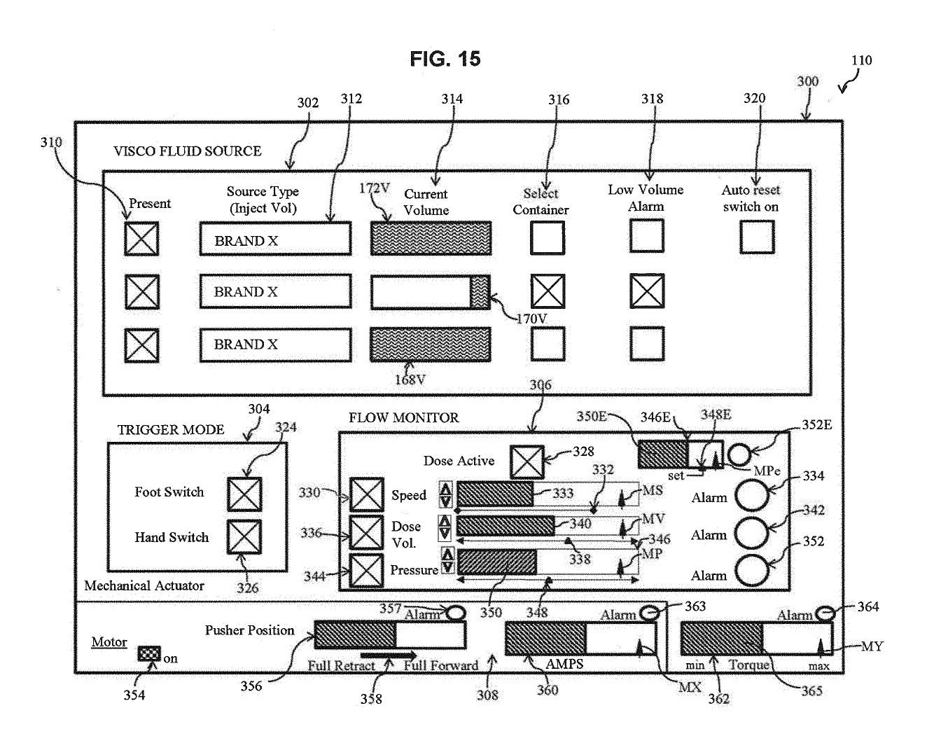

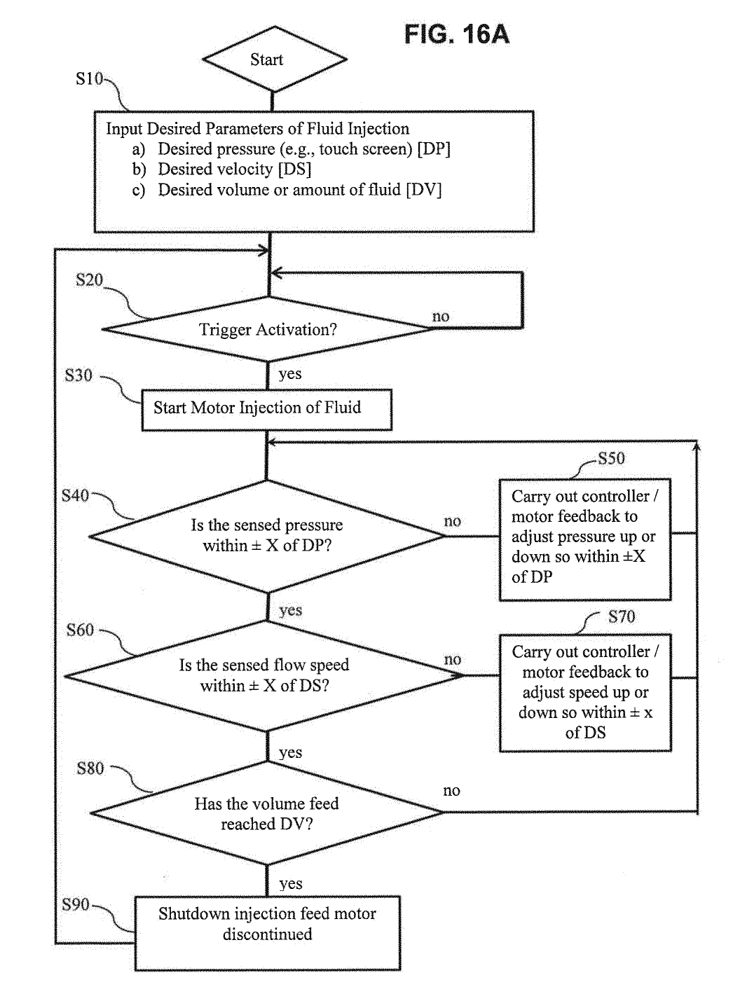

FIG. 15 provides an example of a user interface screen 300 forming part of the user visualization interface means associated with visualization system 110 (the illustrated user visualization means includes in this embodiment electronic signal connectivity (wireless hardwired bus, etc.) such as a system featuring an electrical interface 95 featured in the FIG. 3 embodiment). In a preferred embodiment, however, the controller and motor (driver) interface is located in a location as at a touchscreen interface (an assembly including interface screen 300 features the motor interface including an encoder and amplifier) as to provide for a limited sized material housing 122 that is readily portable.

As seen in FIG. 15, interface screen 300 features four sub-sections; namely, visco fluid source section 302, trigger mode section 304, flow monitor section 306, and mechanical actuator monitoring section 308. Visco fluid source section 302 includes a container information visualization means inclusive of a “container present” information section 310 wherein whether or not a container is present relative to multiple potential container locations is indicated. For example, sensing means as in a visual monitoring (e.g., camera monitoring or a photo sensor arrangement such as blocked light photo sensor) positioned within each of container cradles 174, 176 and 178 with a suitable flexible wire harness to handle the side-to-side movement of the cradles. Alternatively the presence or not of a container prior to a procedure can be input by an operator as by a touch screen toggle or field pressure point following operator visual inspection.

In the embodiment shown in FIG. 15, the presence of a visco material container is displayed (e.g., an LED light “on” for each possible location (i.e., three containers possible and three containers present) is demarcated in FIG. 15). There is also featured in FIG. 15 means for designating the brand of the viscoelastic material to be utilized 312 (e.g., see the earlier designation of the various brands as in Viscoat® and Healon® material which can be sourced from a scroll listing (not shown) of possible brands on the interface of visualization section 110). This information can be input by an operator or based on visual detection or contact detection (as by cylinder diameter monitoring if of different sizes). In FIG. 15, there is shown that three of the same containers of “Brand X” are mounted in respective cradles. As different brands can have different viscoelastic properties these properties can be stored in association with the option choices for ready pull up by the embedded computer (or other processing means) and used appropriately in the flow, speed, and/or pressure calculations.

Visco fluid source section 302 further includes present volume designation section 314 which shows a monitored (virtual presentation) level of the viscoelastic fluid in each container (as by the above described plunger location designation which is known by the position controller system described above). In the FIG. 15 embodiment, there is shown that the central container (e.g., container 170) is nearing an empty state, while the other two containers 168 and 172 are in a full state (virtual depictions of these containers represented by 168V, 170V, and 172V).

Visco fluid source section 302 further includes a “select container” designator 316 which represents an operator input (or an automated selection input) wherein the container to be used for supplying the viscoelastic fluid to the eye chamber is represented. As seen by a review of the current volume designation section 314 and select container designator 316, middle container 170 represents the chosen container and its state is close to empty suggesting a system state wherein it is close to the time to switch over to a new container with a minimum of disruption, inclusive of situations relative to an ongoing procedure (as in a minimum disruption in an ongoing phacoemulsification break up of a cataract in an eye).

FIG. 15 also illustrates low-volume alarm 318 which represents an alarm that is triggered when the current volume level as shown in current volume designation section reaches a lower level (e.g., 10% or less in volume left for dispensing). An alarm triggered by low volume alarm 318 is any suitable conveyance of alarm state information as in visual blinking light as featured in FIG. 15 and/or an audible alarm or both. Such as alarm generation is intended to trigger an operator to manually adjust push-pull member 150 to a new fuller container position, or low volume alarm 318 can also trigger an automatic container adjustment under modes of the invention having an automated version push-pull of adjustment member 150A of adjustment means 199.

That is, FIG. 15 shows the feature of an auto reset switch designator 320 which is suitable in a system having an automated container side-to-side adjustment means such as the above described worm screw or pulley system for adjustment of push-pull member 150 (150A) by an automated process rather than a manual process, and which has been selected for use by an operator as shown by designator 320. If an available automated mode is chosen over a manual mode for a certain procedure, the determination of a low volume setting in a currently utilized container (e.g., upon an alarm level container volume being reached) leads to implementation of a controller signaled container usage adjustment. That is, the controller having the current volume input level information from section 314 is aware of a low-volume situation in central container 170 as well as a full setting situation in either of containers 168 and 172. Accordingly, the controller is configured to automatically adjust, as by an automated push-pull member 150A of adjustment means 199, to position one of the two still full containers in an alignment position with actuator 206 (when the auto reset switch 320 is an active mode).

In situations where an operator prefers non-automated mode as in hand manipulation of push-pull member 150 the auto reset designator can be switched off (as by pressing a touchscreen visual display switch at the location where the on/off designation is located).

Visualization component 110 with visual interface screen 300 further includes trigger mode section 304 which includes trigger designation choices and visual active mode display means. In the illustrated section 304 there is shown two options with an operator foot switch option designator 324 and a hand switch option 326 (e.g., a phacoemulsification handle switch as in the hand piece switch 11 shown in FIG. 1 and/or an alternative foot switch as in foot switch trigger 97 shown in FIG. 1A). In the FIG. 15 embodiment there is illustrated that both options are activated (and presently available in the noted system). This arrangement provides for operator preference as in the option of foot triggering only (e.g., for surgeons who prefer not to have to adjust finger positioning on a phaco tool during an operation and avoid such hand control disruption in favor of foot switch triggering).

Visual interface screen 300 further includes flow monitor section 306 which, in the illustrated embodiment, includes dose active designator 328, which when in display mode is indicative of a visco fluid injection mode being in an ongoing state. That is, when the dose active light display is off there is no injection activity on going (e.g., foot switch in a pause or off state). Further, flow monitor section 306 further includes a speed monitoring on/off state display 330 and an associated preferred or target setting designator 332 and a current speed of visco flow state 333 (an initial activation of the motor with quick ramp up to a feedback monitored state to the chosen target viscoelastic flow speed setting). Target designator 332 is shown below the maximum setting shown in FIG. 15 by maximum speed designator “MS”. It is noted that with the extremely quick ramp up speed provided by a DC motor, when all systems are properly operating the illustrated rates should rapidly approach the preset target level whereupon any excess or failure to timely reach a preset state can trigger an alarm.

For instance, flow monitor section 306 further includes speed alarm designator 334 wherein if the speed exceeds the target value (e.g., an operator set speed value suited for the planned procedure plus a factor of safety positioned a percentage above that level (e.g., 10% over the target set value) an alarm is triggered via alarm designator 334. Also, if the MS value is reached thereafter suitable controller adjustments are made as in a shut off of the motor or a ramping down of electric motor output.

Flow monitor 306 includes similar dose volume active designator 336 and preset operator volume set target input and display designator 338 as well as on-going current visco fluid flow level demarcation means 340 (e.g., a visual display featuring a rising/lowering bar demarcation within the setting range). Further included is an overshoot alarm 342 as in when the flow volume of the injected viscoelastic fluid overshoots a preset value (e.g., a 10% overshoot alarm designation). Further, upon the dose volume reaching a set maximum volume (e.g., unsafe to exceed with a factor of safety range) MV, the controller imitates a shutdown procedure or other safety protocol procedure that is the controller shuts down or lowers pump operation if the MV value is reached).

Flow monitor 306 section further includes similar line pressure active designator 344 showing real-time pressure monitoring of ongoing performance. Also featured is a preset target visco flow line pressure level display 346 with pressure preset display 348 as well as real-time flow pressure display means (as in similar dynamic bar display 350 showing a real-time line pressure level). A corresponding overshoot alarm display 352 and safety shut down maximum pressure MP setting. A similar pressure monitoring set up is also shown in FIG. 15 relative to intraocular pressure, as detected by, for example, intraocular pressure system 3 (FIG. 1), featuring an anterior eye chamber pressure level display 346E, with intraocular preset target pressure setting display 348E, as well as a real-time flow pressure display means (as in similar dynamic bar display 350E showing a real-time line pressure level) and safety shut down maximum pressure in eye setting MPe as well as an associated (sufficiently past target) alarm 352E.

Visual interface screen 300 (with operator-controller interfacing capability) is further shown as including mechanical actuator monitoring section 308 that includes motor on/off designator 354. Mechanical actuator section 308 further includes actuator or pusher position designator 356 shown in this embodiment as a slide level designator using a visual slide bar as in a lit up section marking position of a portion of the plunger relative to a full retract setting and a full forward setting (for center Brand X container showing as being dispensed at 316).

Designator 356 further includes a direction arrow sub-window 358 showing the present direction of plunger movement as in “←” retraction direction (with motor in a “reverse” setting), “→” forward injection direction (with motor in a “forward” setting), and a stop “\textcircled{x})” state designation (with motor in a pause or off state) relative to a present controller implementation strategy. Additionally, shown for mechanical actuator section 308 is electric motor amperage (and/or voltage) display 360 showing an operational state of the motor (an alarm display 363 is also featured in an aspect of the invention). Again, an alarm can be triggered if a first threshold value is reached and a system shut down can be implemented upon a corresponding “MX” value having been reached (e.g., amperage).

Still further shown in FIG. 15 is a torque designator 362 showing (again by a dynamic bar graph 365, although alternate display means are featured as in a dial or a different colored background display such as a green to red demarcation with rotating display needle (etc.)). Such alternate display means is also true for the other visual bar graph display modes shown in display/interface 300. Torque alarm designator 364 is also featured which can provide for system shut down upon reaching a maximum torque “MY” value, as when a torque level rise is suggestive of a flow line blockage or a lack of sufficient fluidity in the visco material being injected.

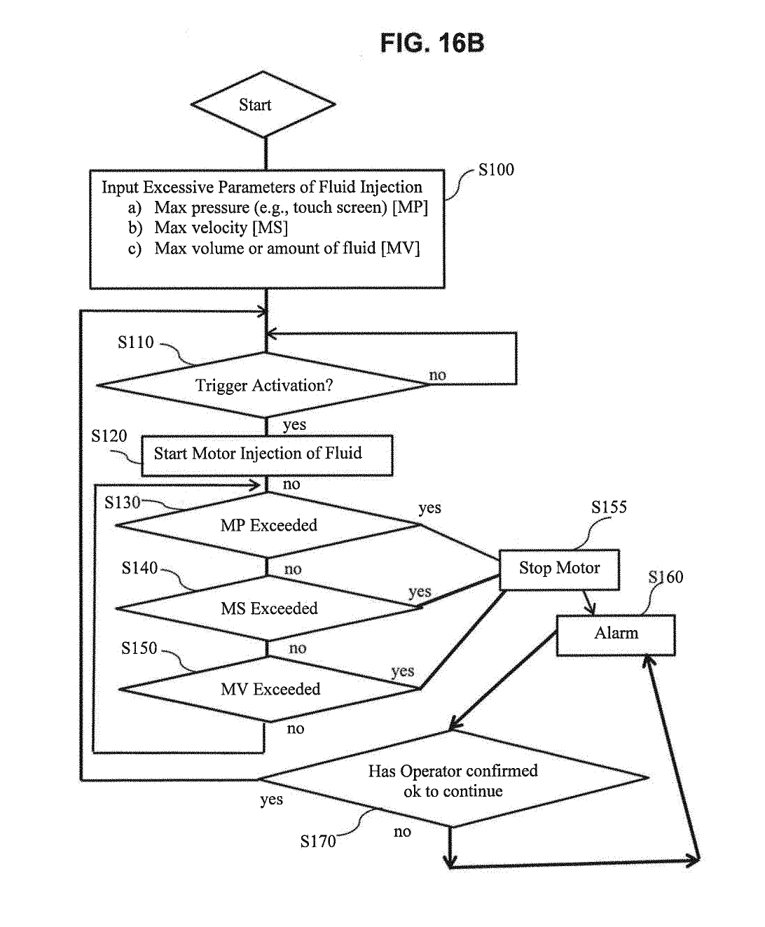

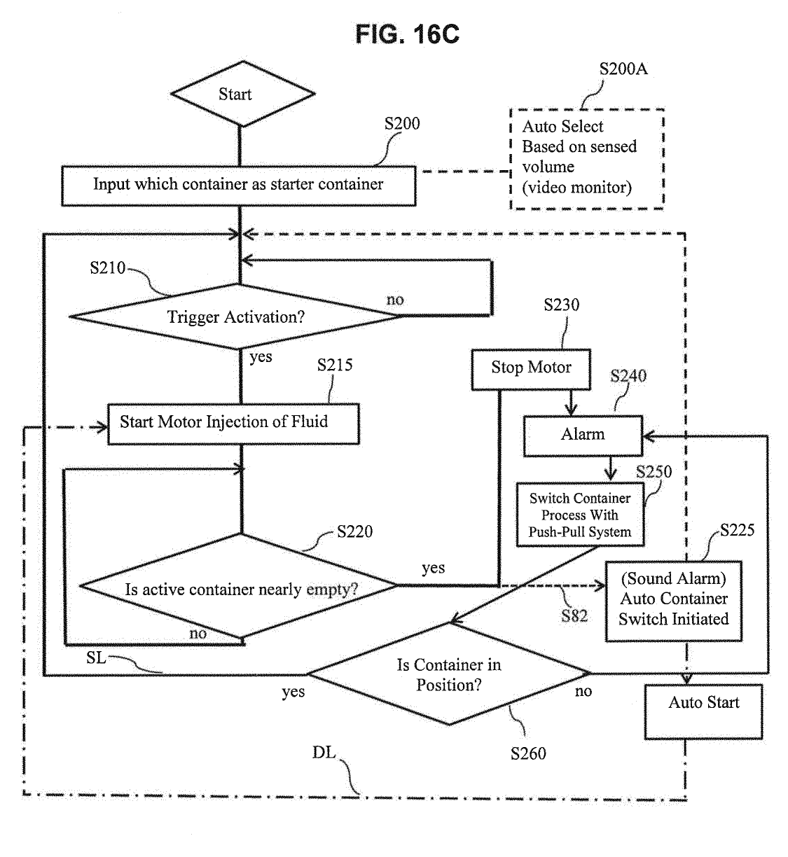

FIG. 16A illustrates a logic flow sequence and operator input step sequence for a method of injecting viscoelastic material aspect falling under an aspect of the present invention. As seen in FIG. 16A, there is Step S10 wherein the operator inputs presets, as in operator desired or “target” fluid flow pressure DP (and/or intraocular eye pressure), fluid flow speed or velocity

DS, and fluid flow volume DV when the system is operating to inject viscoelastic material to the recipient's eye. In this way, the operator (e.g., surgeon or surgeon's assistant) can input desired levels based on the aforementioned criteria as in age, health, eye size characteristics, etc. These inputs can be put in by an operator via a visual interface, as described above in the FIG. 15 discussion, or some other input means.

Following settings having been input by the operator, the system reviews at step S20 whether trigger activation has occurred, as in a foot pedal activation. If no, the system goes back into a repeating monitoring mode as to whether the trigger has been activated. If a trigger start request has been activated and the controller determines there is no obstacle to start up (e.g., an alarm mode is not active) the injection system is started by starting the motor at step S30 which in turn drives the downstream transmission system TR (e.g., a linear screw drive as shown). Also, the sensor components of the system monitor for whether or not the system has reached the input desired (target) parameters.

For example, as shown in FIG. 16A a review is made at step S40 as to whether the current measured pressure is within a target DP range. That is, the controller receives flow pressure input and compares the sensed value against a pre-input value DP, or more preferably a +/− range (e.g., 10%) of the set value DP to help absorb some natural system fluctuations. If the value is not within the noted DP range, under step S50 (after a suitable ramp up time period accommodated in the logic consideration) a determination is made by way of the communication of the one or more pressure sensor feeds to the controller (e.g., intraocular pressure monitoring system 8 in FIG. 1) and the controller determines in a feedback loop with the motor whether to enhance or retract the motor rate (or start/stop the motor) to increase or decrease the flow pressure. Upon such an up or down adjustment, a return is made to the DP range check step S40. Also, if the controller deems the pressure is above the noted DP range a further logic review can be made as to whether a maximum system pressure has been reached as per the below discussion directed at FIG. 16B (in similar fashion if a set pressure is not reached in a predetermined time a warning signal or shut down can be activated).

Upon a controller determination that the considered pressure (e.g., in eye and/or in line pressure monitoring relative to one or each input pressure value range) is within the noted DP range at step S40, the process continues to flow speed monitoring step at S60 (shown in series with the above pressure review) although aspects of the invention include parallel reviews as well, or the removal of one or two of the pressure, speed and volume level monitoring fields.

In step S60 a similar sensor input and controller comparison against the input (target) flow speed value DS, or more preferably a +/− range relative to DS (e.g., +/−10% of set value) is carried out. If the value is not within the noted DS range, under step S70 a determination is made by way of the communication of the one or more flow speed sensor feeds to the controller (e.g., sensor 4 in FIG. 1) and the controller determines in a feedback loop with the motor whether to enhance or retract the motor rate (or start/stop the motor) to increase or decrease the fluid flow speed to the eye. Following the adjustment made at step 370 a return to step S40 is made to check to see if both pressure and speed are within desirable limits (or a parallel, both met, move on sequence can be implemented—not shown). Again, if a predetermined time passes without reaching a desired setting, a warning and/or shut down sequence can be implemented.

Once it is deemed that the pressure and flow speed are acceptable, a review is made at step S80 as to the current volume amount that has been fed since trigger initiation in the system. If the volume has reached a predetermined level, the injection feed motor is shut down at step S90, and a return is made to a location upstream of the trigger activation review step S20. The volume measurement made at S80 can be made with the assistance of the controller and a viscoelastic material volume measurement means such as the above described injection measuring device 4 in FIG. 1. Preferably a corresponding signal is made as to the preset or desired volume injection to the eye having been reached as in the visual display described in FIG. 15. The reinitation of volume feed can be tied in with the sensor control review of, for example, the intraocular pressuring in the eye chamber, which upon dropping to a lower value setting can trigger a volume plug feed with the viscoelastic feed means of the present invention.

FIG. 16B illustrates an additional logic flow system, which like the steps described above in FIG. 16A, can be a subroutine in any one of the above described controllers with, for example, sensor inputs and processor parameters to determine the next step sequence. Although FIG. 16B is shown as an independent controller subroutine, as noted above, there can also be coordination as in once the above described pressure, speed and volume level reviews, carried out with respect to DP; DS; and DV suggest an overage there can be initiated a “MAX” check based on that sensed overage determination information. That is, with a determination in any of steps S40; S60 and S80 of an above level finding there can be a further check as to whether any of the below described maximum values MP; MS and MV has been reached. Alternatively the same or different sensors for pressure, flow speed and viscoelastic material volume supply (or a subset of the same as in pressure only) can further be checked in an independent routine from that featured in FIG. 16A.

In FIG. 16B there is input or preset the three parameters MP, MS and MV, but a subset or single one can be only checked if deemed sufficient informative of a system problem.

FIG. 16B shows an operator input step (in this embodiment) all three parameters MP; MS and MV can be input by the operator, with S100 wherein any level above the max values being considered an indicator of system problems, and a need to warn or take corrective action. For example, with respect to intraocular eye pressure, a maximum typical value experienced in various operations is at about 110 Hmg. Thus, a maximum pressure of, for instance, 10% above 110 Hmg for a healthy patient (or a lower % or no increase, or even a lesser than 110 Hmg value may be set as in for a patient deemed not healthy). For example, 120 Hmg can be set for a healthy patient with a lower value set (e.g., 110 Hmg for a less healthy patient or one with other issues suggesting a lower setting based on operator input decisions). Following desired input settings (or retention of default setting if nothing input), the logic flow of the associated controller moves to step S110 for a determination or not whether the operator has triggered for viscoelastic material injection. If such a triggering event has occurred, there is initiated injection flow (suitable coordination as to pump start can also be made relative to the routine of FIG. 16A as in a common sensed trigger activation signal) at step S120. Once that motor is activated for injection, sensing is undertaken at each of steps S130, S140 and S150 (shown in sequence although parallel sensing can be undertaken) for the respective maximum values for MP; MS and MV. If any of the maximum values are reached, there is initiated a stop injection (e.g., stop pump motor source) at step S155 in conjunction with an alarm signal generation (e.g., sound and/or visual) at step S160.

In FIG. 16B there is further shown a lock out release step S170 wherein the processor system check is made at step S170 to determine if the operator has confirmed by way of an operator input (button press) that confirms that the system is suited for restart, whereupon if the confirmation is received the system returns to checking for whether the trigger activation has been undertaken at step S110. The sensing for maximum value routines then continue while the system is operating to inject viscoelastic material. Also, the overpressure shut down monitoring is preferably carried out for both overpressure in the line pressure and the measured anterior eye chamber pressure as determined by, for example, intraocular pressure monitoring system 8 (i.e., if either one of the pressure levels reaches a maximum value the pump is shut off and suitable warnings made).

FIG. 16C illustrates a flow logic sequence as a controller component under any of the pump injection systems described above where there is multiple viscoelastic container sources such as that featured in FIG. 5. At step S200 an operator inputs which container of the multiple containers is intended for initial use upon trigger activation. This can be determined by way of an operator reviewing the virtual container level fill depictions discussed above in the FIG. 15 discussion. Alternatively, when there is present an automated container adjustment, there can be an automated initial use container determination and adjustment into position, if needed, based on the sensed virtual fill levels. In either situation, after the desired container is positioned for initial use, a review as to trigger activation is made in step S210 which if activated initiates the above described container injection usage (start pump motor and monitor fill levels with virtual depictions signaling) at step S215 (e.g., as by way of the encoder monitoring associated with the motor and/or transmission component that is driving the actuator to push the plunger to eject viscoelastic material). This sensing provides for the logic determination shown at step S220, wherein a review is made as to whether the currently used viscoelastic container is near or at empty as to require container switching to provide for continued operation. If upon a determination at S220 of yes (container deemed empty) there is implemented a stop (e.g., pause) motor (or injection source driver) step S230, preferably in conjunction with an alarm generation at S240.

At this stage the signal alarm at S240 informs the operator to implement a container switch at step S250, which, in an aspect of the present invention, is easily implemented by way of the adjustment using push-pull member 150 (operator sliding one way or the other to place a fuller container in position with, for example, reference to the virtual fill levels provided with user interface screen). For example, using the controllers check based on the known relative position of the actuator (see pusher position designation of pusher position designator 356 in FIG. 15), there is known the fill state of the containers. Stated differently, if the pusher has reached the near full forward position and the container is known to be nearing empty and in need of switch over, there is implemented a stop (pause) motor step S230 and a signal is generated to the operator as to manual switch over need at S240. Thereupon, in the non-automated version of the present invention, there is implemented by the operator a manual switch over using push-pull member 150 to position a fuller container in alignment with the actuator (the display is then updated as by the known sequence of the actuator longitudinal forward and back motion location such that an updated display designation as to the fill levels of the various containers present in the system is made). After the signal to switch containers is implemented for the operator to manually provide a fuller container into the actuation position, a check is made to confirm that a container is in position at S260, and, if so, the monitoring returns to step S210 to determine if a restart trigger activation has been implemented by the operator.

FIG. 16C also shows in dashed lines at S82 an automated version of the above described manual slide adjustment to line up a new (fuller) container with the actuator. Under this arrangement, wherein there is an indication that there is a need to implement a container switch over at S220, the controller initiates an automated routine relative to dashed signal line S82, wherein there is preferably an operator informative signal generated to let the operator know that there is a fuller container switch over in process. Under such an automated container switch over, a review is made by the controller (in conjunction with the sensed status of the containers) whether a suitable fuller container is available and, if so, there is triggered auto container slide means or adjustment means 199 (e.g., the aforementioned worm gear and pinion drive shown auto push-pull means 150A in FIG. 7) to appropriately position that fuller container.

In FIG. 16C the solid line SL shows a return to a trigger switch determination step S210 wherein the system waits for operator input to continue injection with the newly positioned, fuller container. FIG. 16C also illustrates by dash-dot line DL an automated restart of the motor or injection driver start without a requirement for operator initiation (or a trigger activation check can be made as shown in dashed lines if such a check is desired before restarting from a previously inject-on status being in place before there was sensed a need to switch over containers).

Also, whether an automated or manual switch out, the motor is preferably left in a power on but no drive (or pause) mode to enable a rapid restart following switch out completion.

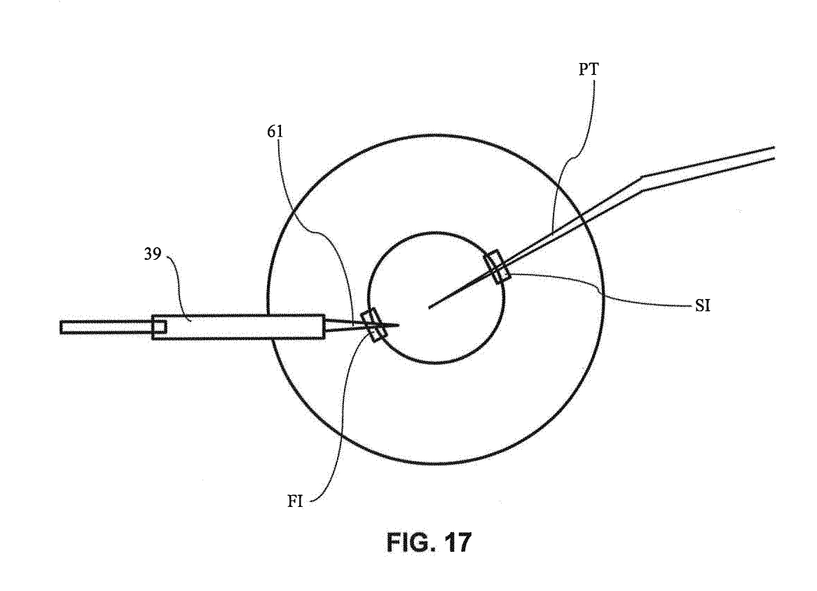

FIG. 17 shows an example of the positioning of the needle 61 of viscoelastic maintainer 39 received in a first incision FI provided by the operator. FIG. 17 also shows the phaco tool PT at the end of handpiece (phaco needle preferably having a co-aligned combination aspiration/saline supply tube such as represented by tube 27 in FIG. 2) is provided in a second, independent incision SI in the eye. Using this combination and any of the viscoelastic injection embodiments described above there can be supplied a desired amount of viscoelastic material through needle 61 which helps both maintain eye shape (despite the phaco vibration, the aspiration of material out, and the injection of saline to the cataract eye cavity being operated upon) while also providing a protective coating to sensitive tissue as in the endothelial cells.

The present invention also features methods of operation including the above described steps involving manual input in FIGS. 16A to 16C inclusive of screen 300 target set inputting as well as manual switch over of non-full container as described in the non-automated portions of FIG. 16C. In addition, a method of the invention includes the formation of the above described first and second incisions and the insertion of the maintainer 39 and phaco tool PT into the respective incisions and adjustment of the phaco tool (physically and controlling flows/vibrations) while simultaneously controlling flow of visco material to the eye through maintainer using the activation trigger means described above such as the noted foot pedal.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but it is to be understood in the broadest sense allowable by law.

What is claimed is:

1. A system for delivering viscoelastic material to an eye, comprising:
a driver,
a transmission in driving communication with the driver,
an actuator in driving communication with the transmission, and
a viscoelastic material support assembly which includes a multiple viscoelastic material container retention supporting structure and an adjustment mechanism for adjusting the retention supporting structure relative to the actuator into an injection capable setting.

2. The system of claim 1 further comprising a controller to activate the driver upon the adjustment mechanism aligning one of a plurality of container supports of the retention supporting structure into the injection capable setting.

3. The system of claim 1 wherein the adjustment mechanism includes a manual push-pull member connected with the container retention supporting structure.

4. The system of claim 3 wherein the container retention supporting structure includes a plurality of cradles each configured to receive a respective one of a plurality of viscoelastic material containers.

5. The system of claim 4 wherein the cradles are dimensioned to receive syringe shaped viscoelastic material containers having plungers extending out from fluid containing cylinders.

6. The system of claim 3 wherein the retention supporting structure includes a plurality of support blocks that include slide tracking for side-to-side adjustment based on positioning of the push-pull member.

7. The system of claim 1 wherein the retention supporting structure includes a plurality of support blocks that include slide tracking for side-to-side adjustment based on positioning of the adjustment mechanism.

8. The system of claim 7 wherein each support block has a universal configuration relative to another support block of said support blocks.

9. The system of claim 1 further comprising a housing wherein said housing includes a slide track on which the container retention supporting structure slides.

10. The system of claim 1 wherein the actuator includes a base part and a projection part wherein the projection part is configured for abutment with a plunger portion of a viscoelastic material container aligned by the adjustment mechanism for emptying.

11. The system of claim 10 wherein the transmission includes a linear screw assembly to which the actuator is movably connected.

12. The system of claim 1 wherein the actuator is adjustable in a longitudinal direction transverse to the side-to-side slide direction of the container retention supporting structure.

13. The system of claim 1 further comprising a controller in position communicating with the actuator, and said controller having an actuator position monitoring unit that monitors actuator position and conveys that information indicative as to container fill level to the controller.

14. The system of claim 13 further comprising a visualization system in communication with the controller, and which visualization system has a fill level designation section that conveys a fill level of one or more of the containers.

15. The system of claim 14 where in the fill level designation section conveys the fill level of each of multiple containers supported by the container retention supporting structure.

16. The system of claim 1 further comprising a controller and wherein the driver is an electric motor that communicates torque level data of the motor and conveys that torque information as to pressure levels of viscoelastic fluid being fed by actuator movement by the actuator.

17. The system of claim 1 wherein the retention supporting structure includes multiple cradles for supporting viscoelastic containers which are emptied in a longitudinal direction, and wherein the retention supporting structure includes base blocks which support said cradles and each include a slide configuration for sliding in a side-to-side direction which is perpendicular to the longitudinal direction.

18. A system for delivering viscoelastic material to and eye comprising:
driving means for providing driving forces,
an actuator which receives the driving forces,
supporting means for supporting a plurality of containers,
adjustment means for adjusting the supporting means to multiple positions in a desired container in position for actuator adjustment.

19. The system of claim 18 further comprising a visualization system showing which container is adjusted for immediate dispensing and the relative fill level for each of the multiple containers.

20. The system of claim 18 further comprising a visualization system showing the relative fill level of the multiple containers and the adjustment means being adjustable to place a more full container in place of a less full container in alignment with an actuator of the actuation means.

21. A method of delivering viscoelastic material utilizing the system of claim 1, comprising adjusting the retention support structure as to move a less full viscoelastic material container from an actuator alignment position to another location while bringing a more full viscoelastic material container into the actuator alignment position.
